SEQUENCE LISTING (9 sequences)

SEQ ID NO: 1 - protein, 1110 aa, Oocystis sp.
  source 1..1110: note = PKL-0924 kinase-like protein wild-type sequence
MRDPSGSATRPTPARVHLARDPSPCKPMPQDFSVSDQQGNGAFDAPDAAGAACTTLPRRSSGAHDCRDMVAQEHGDGAAQVAQPSQTSAVSKMTHELLATYQRCGGGVGPTQASDATHALHQLQQANQAPEQRQQAQRNLLTKPFKGLHNDGHDNENWDLIICVGDEFVSSSNLKYVVIDLLGQGTFGQVVRCWCDQTQEYVAVKVIKNQPAYYQQARVEVGLLQYLNRCADADDVRHIVRLRDYFLFRNHLCLAFELLSVNLYELIKHNQFRGLSAGLVRVFIAQLLDALVVLRESRLIHCDLKPENVLLTGAESADIKVIDFGSACLESKTVYSYIQSRFYRSPEVVLGYPYNVAIDMWSLGCMAAELFLGLPLFPGASEHDLLSRVVQAVGLPPLYLLQGAKHTNKYFKMVERVVRLPSGRSEVVPEYVMRTAAEFEALTGLKATTGKRYFSHTRLQDIINSYPSEGAGSELRRSLLDFLRGVLDPDPAARWTPQQAARHPFVTGQPFAAPFQPDGERTPPPPGWDASAAGMLSSSAGHQAAQQQQAGWNGASASPHYGSAAAAMLATSPHVQAHVAAMAALAQQQQQQGMGTPSGLPSYRPQPVPVPRAGPGYGGGGGWAAPYPHQHQQQQQAGSLTQQYLQQQQQQAGGSLGLFSPPGLGPASLMQLDHLSAASGSFFSPPGSLVNATGRLAPAPGAARYGSYQPMPVDMGFSPAGSLSANSVLAVAIAAANAAAASAAAQQQQQQHGVAEQLRLQWQGAAVGGGGGGGFPSGLARMSGVAGGSYSGQSAVPMVGSYASSLPGGSAATAAAGPGTGSNPLAMLAAQQAAEDALASSLRRGSMGAGASGGYGAGFGGSAAATAAPAAAAAAAQQPAPVDMSAAAGSLGAVLQQLQATQAAAQAQQFVAAAAGGGSRGRTPPPPADGALATACAAAAAGAVAGGGGSAHQQQQRHESVSPSPGDWDPLYSDDQLLEDDTPAQQRTPPQQPQQRKGSGNLAAALAAAVTLPAQQQQWQQQQQQRQPLAGGLSPPDPALTAAAAAALGALQQQQQQQQATAGAGAATLDTADLVGWLQQQQLLRALRPPPATADARGAGGAAAAAARLH

SEQ ID NO: 2 - DNA, 3751 bp, Oocystis sp.
  source 1..3751: mol_type = genomic DNA; note = PKL-0924 kinase-like protein domain wild-type cDNA (exons only) 30070, encodes SEQ ID NO: 1
atgcgcgatcccagtgggagtgccacgcgacctactcctgctcgggttcacctggcggggaccccagcccatgcaaaccaatgccccaggatttcagcgtgtctgatcagcaggtaacggcgcctttgacgcgcctgatgccgctggagctgcgtgtacgacattgccgcacgatctagtggcgctcacgactgcagggacatggtagcccaggaacacggagacgggccgcccaggtggcacagccatcccagacatcggctgtgtccagtagttcgtgtgtatcttcgccacgtatcaacggtgcggtggaggcgttggaccgacacaagccagcgagcaacgcacgcgttgcatcagctgcagcaggccaatcaagcaccggagcagcggcacaggcacagcgcaatttgctcacgaagcctttcaagggtctgcataacgacggtcatacaacgagaactgggacttgatcatatgtgtcggggacgagtttgtgtccagctccaacctcaagtacgtcgtcatcgacttgctgggccagggcacgttcggccaggtagtgcgctgctggtgcgaccagacgcaggagtatgtagccgtcaaggtgataaaaaatcagccggcttactatcagcaagcgcgcgtagaggttggcctgttgcaatacctcaaccgctgtgcggacgcggacgacgtccggcacattgtgcgcctccgcgactactttttgttccgtaaccacttgtgcctcgcgttcgagctgctgtcggtcaacctgtacgagctcatcaagcacaaccagttccgtgggctgtctgcggggctcgtgcgcgtgttcatcgctcagctgcttgatgcgctggtggtgctgcgtgagtctcgcctcatccactgcgacctcaagccggagaacgtgctgcttacgggcgctgagtcagctgacataaaggtcatcgactttgggtccgcttgcctggagagcaaaacggtgtacagctacatccagagccgcttctatcgctctccagaggtggtgcttggctacccgtacaacgtggcgatcgacatgtggtccctgggctgcatggccgcggagctgttccttggcctgccgctgttcccgggcgcatcggagcacgatttgttgtctcgggtggtgcaggcggtgggcctcccgccgctgtacctgctgcagggcgcaaagcacaccaacaagtatttcaagatggtggagcgcgtggtgcggctgccaagcggcaggtctgaggtggtgcccgagtatgtgatgcgcactgccgcggagtttgaggctctcacggggctcaaggccaccaccggcaagcgctacttctcacatacgcgcctgcaggacatcatcaactcatacccttcagagggcgcgggcagcgagctgcgccgctcccttctggacttccttcggggcgtgctggaccccgacccagcggcgcgctggacgccgcagcaggcggcgcgccacccgtttgtgacgggccagccgttcgcggcgccgttccagccagacggggagcgtacgcccccgcctcccggctgggacgcctccgcggccggcatgctgtccagcagtgctgggcaccaggccgctcagcagcagcaggcagggtggaacggcgcgtccgcgtcgccgcactatggcagcgcggcggcggcgatgctcgccacgtcgccgcacgtgcaggcgcatgtggcggccatggcggcgctcgcccagcagcagcagcagcagggcatgggcaccccgtcagggctgccctcgtaccgcccgcagcctgtgcctgtaccgcgtgcggggcccggctacggcggtggcggcggctgggcagcgccgtatccccaccagcaccagcagcagcagcaggccggcagccttacgcagcagtacctgcagcagcagcagcagcaggcaggcggcagcctgggcctgttcagcccgcccggcctgggccccgccagcctgatgcagctcgaccacctgagcgcggcctcgggctcgttcttcagcccgcctgggtcgctggtcaacgcgaccgggcggctggcgccagcgccgggggccgcgcgctacggcagctaccagccgatgccggtggacatgggcttcagccccgccggcagcctgtcggcaaactcggtgctggcggtggccatcgcggcagccaacgccgcagccgcctcggccgctgcgcagcagcagcagcagcagcacggcgtcgcggagcagctgcggttgcagtggcagggcgccgccgtgggcggcggcggcggcggcggcttccccagtgggctggcgcgcatgtcgggcgtggcagggggctcgtactccggccagtctgcagtgccgatggtggggtcgtatgccagcagcctgccggggggcagcgccgccaccgcggcggcgggcccaggcacgggctcaaacccgctggccatgctcgcggcgcagcaggcggccgaggacgcactcgccagcagcctgcggcgcgggtcgatgggcgcaggcgcgagcggcgggtatggcgccggcttcggcggctccgcagcggccacggccgctcccgccgccgccgcggcggcggcccagcagcctgcgccggtggacatgtcggctgctgcaggcagcctcggcgcggtgctgcagcagctgcaggcgacacaggccgcggcgcaggcgcaacagtttgtggcggcggccgccggtggcgggagccgcggccgtacgccgccgccgccggcggacggtgcgcttgccaccgcttgcgctgcagctgctgcgggagcagtggccggggggggcggcagcgcccaccagcagcagcagcggcacgagtcggtgtcgccatctcctggggactgggacccgctgtacagcgacgaccagctcctggaggacgatacgcccgctcagcagcgtacaccgccgcaacaaccgcagcagcgcaagggcagcggcaacttggcagcggcgctggcagcggccgtcacgctgcctgcccagcagcagcagtggcagcagcagcagcagcagcggcagcccctcgcgggcgggctgtctccgccagaccctgcgctaacagcggcagccgcggcggcacttggcgcgctgcagcagcagcagcagcagcagcaggccacggcaggtgccggtgcggccacactggacaccgcagacctagtgggctggctgcagcagcagcagctgctgcgcgcgctgcgcccgcctccggcaacggcagacgcccgcggcgccggaggtgccgccgctgccgcggccgcctccattgagcctgctggtgtggcgggtgccgtgtcggcggcggcgccggtgcccggcgaccaacagcaccgtgcagcagggccaatgccgcccctctctgctgccgagcagcagcaaacacaggccggtagcagcgggggcgctgctgcccatgctgcggcagcagcaggggaaggtgttgccgccggcctgggcggcggtgctggtggcgcctctcggcggaacagttttgagattgatcggaggggtagttttgagattggctttgaggttgctccagtcgtgtccacaggcctgcagcaggcgtcgggagcgcgcactggcgcggcacccgcccacgccgggggtggattcagctgcttgtctgcgcagctccacggcagcaggcagcacaagcagcagcagccggaagcggcagaacattccagcaagtag

SEQ ID NO: 3 - DNA, 6872 bp, Oocystis sp.
  source 1..6872: mol_type = genomic DNA; note = PKL-0924 wild type genomic DNA (including introns) encodes SEQ ID NO: 1
atgcgcgatcccagtgggagtgccacgcgacctactcctgctcgggttcacctggcgcgggaccccagcccatgcaaaccaatgccccaggatttcagcgtgtctgatcagcagggtaacggcgcctttgacgcgcctgatgccgctggagctgcgtgtacgacattgccgcgacgatctagtggcgctcacgactgcagggacatggtagcccaggaacacggagacggggccgcccaggtggcacagccatcccagacatcggtgagaagacgcccggcatgcctatcttatcgcttgcgtgtgcgccagcgcgtcgcagtgccctggttgggtgctcgtcgcgctgtgactatcggtacttgagcgttttataaccggcacgcacgtgcttgtcgttgatgcgttgtgtaggctgtgtccagtagttcgtgtgtcatcttcgccacgtatcaacggtgcggtggaggcgttggaccgacacaagccagcgacgcaacgcacgcgttgcatcagctgcagcaggccaatcaagcaccggagcagcggcagcaggcacagcgcaatttgctcacgaagcctttcaagggtctgcataacgacggtcatgacaacgagaactgggacttgatcatatgtgtcggggacgagtttgtgtccagctccaacctcaagtaagtccacccccatcaacctcttaaccaactctgcctagcgttgtcaaagtgcttatacatgcagcgcgccgcaacgcgacgagcttattcgctcgttaagtaaatttttctgctgctcgcatcgtatcggttaaccatccgatagctgactgcttccctttctctgtatcgacgggcgcaggtacgtcgtcatcgacttgctgggccagggcacgttcggccaggtagtgcgctgctggtgcgaccagacgcaggagtatgtagccgtcaaggtgataaaaaatcagccggcttactatcagcaagcgcgcgtagaggttggcctgttgcaatacctcaaccgctgtgcggacgcggacgacgtccggcacattgtgcgcctccgcgactactttttgttccgtaaccacttgtgcctcgcgttcgagctgctgtcggtcaacctgtacgagctcatcaagcacaaccagttccgtgggctgtctgcggggctcgtgcgcgtgttcatcgctcaggtgagcacaggaatggattagcagcacaccgccaagttgtgcagtggcgtttggcggggacgctcacgagcagtatcagcagataggcacgaagctagccaacttcaagcgggttggaacctgcccgcctgcagcagtatgcctgccttgccctcccgctgtcgtatcggcgcgttcacccctgcatcctgcacctcccacctatgagtctgcacctcccaaacccctcacactcctctctctctctctctctctctctctctctctctctctctctctctctctctctcgcgtgctatcccctcccttcacatacgcacacatgcacgcgcatacacgatgccacacacacacacacacacacacacacacacacacacacacacacacacacacacacacgcacacatgcactgcaacctgtcacatgcacgtgcgtgcatgtgcagctgcttgatgcgctggtggtgctgcgtgagtctcgcctcatccactgcgacctcaagccggagaacgtgctgcttacgggcgctgagtcagctgacataaaggtcatcgactttgggtccgcttgcctggagagcaaaacggtgtacagctacatccagagccgcttctatcgctctccagaggtgcgcgcgcgcggccatgggtcgacatatggagttgggctttggaaactgctggcactgacaggcttcgggaccaccacggggctgttgttgttcggcagcgtggtgccttcaggggcggtggggtgggagcctggtgctgggagcaggggaggcagagctgattccagtgggcagacccgattccagtgctcgccgcaaggcgacggctacagctggtggctgttgcgcgagttgcaaatgcatgtgcatgtgcacgtgaagcgcacgcccacgcgtaagcatggcacacgcgcgcacgtagacacgcacgcacgcacataccatatcaccaccaactcccaaaggtggtgcttggctacccgtacaacgtggcgatcgacatgtggtccctgggctgcatggccgcggagctgttccttggcctgccgctgttcccgggcgcatcggagcacgatttgttgtctcgggtggtgcaggcggtgggcctcccgccgctgtacctgctgcagggcgcaaagcacaccaacaagtatttcaaggtgcgaaagagtgcgtggggcgggcacgccgtcgatgcgttgccacgccgaggctggcactttctgaatgccgaagcagtcagccataagcttacctgaaagggatgaatggggaagctggtggcccgagcgcactgacaaccacctgtgcggtgaaggttgcgtgtgcgcatgcgcgtgtgggtgcgtggagggcacaggtgcgtgcaagacaggggtggggtggggggcagtgcagtgcaaggccttgcctgcgccctcgccacgtcacgtcaccccacccaacccaacttcgtcaccgcttcgtatgcacacgcatgcacagacacacgcactgaattcaaacgcatgtgcgcatgtttcagatggtggagcgcgtggtgcggctgccaagcggcaggtctgaggtggtgcccgagtatgtgatgcgcactgccgcggagtttgaggctctcacggggctcaaggccaccaccggcaagcgctacttctcacatacgcgcctgcaggtgggcggcgtcaagtgtctcatgcgtgtggcactgtgtgtgtatgacgctgtgtatgcgtgcatgcgtgtgtgcgttcatgtgtgtgtgtgtgcgcgcgcgtgtgcgtgcgtgcgtgcgtgcgtgggtacatgcacgggtacacgtgtgcatttttgtgcgtgtttgtgtgtgtgtgtgacgtgctgttccctgcttgtaatgcagctgaaaggggcgctcatgaggggtagacgatcggcgcactgtgacgccgcgctccttgtcttagaaatcgcgcagcggtggacctcctttgtgcagcgcttgaaacaacgtgccactcgccacctcgctgagactggatttccattgtcacaactccctcagtgacccgtgacccgccagacacgacacccgacctggcggcaccccgcgcgactcacccacgcgcaccaccaacgtcgcctttgccaacgttgcgttcgtatccacaccctacctgatggccatcaccgcgcgcatgcatggcgaagggagctaatccaagccacctcaccctgctccccacaggacatcatcaactcatacccttcagagggcgcgggcagcgagctgcgccgctcccttctggacttccttcggggcgtgctggaccccgacccagcggcgcgctggacgccgcagcaggcggcgcgccacccgtttgtgacgggccagccgttcgcggcgccgttccagccagacggggtgagaggggaggggctgctgggcggggtgagatgggaggggggggggggctgctgggcggggtgagatgggaggggggggctgctgggcggggtgagatgggaggggggggctgctgggcggggtgagaggggagggaggggctgctgggcggggtgagaggggagggaggggctgctgggcggggtgagaggggagggaggggctgctgggcggggtgagagggggggaggggggggcggctgggcggggtgagaggggcctgctgggcggggtgaggggagggggggggcctgctgggcggggtgagaggggagggggggggggctgctgtggaactgccaggccgtgggtagcagcgtgtgtgttgtctgcggtgcttgcggctgtgtaagggcaagcgagagggtgcacttatgtcttcggtgctcctcgcacgggacgcacacgcgcgcaagcatggtgcaaacttacctaccagctggccaagcgtaacgtcatgctgccctaagcatggcggcccctatctgctactgctgctatatcccgtccctgcaggagcgtacgcccccgcctcccggctgggacgcctccgcggccggcatgctgtccagcagtgctgggcaccaggccgctcagcagcagcaggcagggtggaacggcgcgtccgcgtcgccgcactatggcagcgcggcggcggcgatgctcgccacgtcgccgcacgtgcaggcgcatgtggcggccatggcggcgctcgcccagcagcagcagcagcagggcatgggcaccccgtcagggctgccctcgtaccgcccgcagcctgtgcctgtaccgcgtgcggggcccggctacggcggtggcggcggctgggcagcgccgtatccccaccagcaccagcagcagcagcaggccggcagccttacgcagcagtacctgcagcagcagcagcagcaggcaggcggcagcctgggcctgttcagcccgcccggcctgggccccgccagcctgatgcagctcgaccacctgagcgcggcctcgggctcgttcttcagcccgcctgggtcgctggtcaacgcgaccgggcggctggcgccagcgccgggggccgcgcgctacggcagctaccagccgatgccggtggacatgggcttcagccccgccggcagcctgtcggcaaactcggtgctggcggtggccatcgcggcagccaacgccgcagccgcctcggccgctgcgcagcagcagcagcagcagcacggcgtcgcggagcagctgcggttgcagtggcagggcgccgccgtgggcggcggcggcggcggcggcttccccagtgggctggcgcgcatgtcgggcgtggcagggggctcgtactccggccagtctgcagtgccgatggtggggtcgtatgccagcagcctgccggggggcagcgccgccaccgcggcggcgggcccaggcacgggctcaaacccgctggccatgctcgcggcgcagcaggcggccgaggacgcactcgccagcagcctgcggcgcgggtcgatgggcgcaggcgcgagcggcgggtatggcgccggcttcggcggctccgcagcggccacggccgctcccgccgccgccgcggcggcggcccagcagcctgcgccggtggacatgtcggctgctgcaggcagcctcggcgcggtgctgcagcagctgcaggcgacacaggccgcggcgcaggcgcaacagtttgtggcggcggccgccggtggcgggagccgcggccgtacgccgccgccgccggcggacggtgcgcttgccaccgcttgcgctgcagctgctgcgggagcagtggccggggggggcggcagcgcccaccagcagcagcagcggcacgagtcggtgtcgccatctcctggggactgggacccgctgtacaggtactgcctcccaccaccttcacccgttcacgccactgccatgcctccatctgcgtgcctcaggctgccgtcgggacgcgcacatacatgtgtgctgcaaggggctggctagtccaagcgtaaacgtcattactgatgcttgtaccactcaatggatcgtatgactactacgtcagacaatacacccccaggcgctgttcccatacccgtaagcctgacattcaaacgggatgctgacgttgccgcactccctcttcctctttccccaccccacagcgacgaccagctcctggaggacgatacgcccgctcagcagcgtacaccgccgcaacaaccgcagcagcgcaagggcagcggcaacttggcagcggcgctggcagcggccgtcacgctgcctgcccagcagcagcagtggcagcagcagcagcagcagcggcagcccctcgcgggcgggctgtctccgccagaccctgcgctaacagcggcagccgcggcggcacttggcgcgctgcagcagcagcagcagcagcagcaggccacggcaggtgccggtgcggccacactggacaccgcagacctagtgggctggctgcagcagcagcagctgctgcgcgcgctgcgcccgcctccggcaacggcagacgcccgcggcgccggaggtgccgccgctgccgcggccgcctccattgagcctgctggtgtggcgggtgccgtgtcggcggcggcgccggtgcccggcgaccaacagcaccgtgcagcagggccaatgccgcccctctctgctgccgagcagcagcaaacacaggccggtagcagcgggggcgctgctgcccatgctgcggcagcagcaggggaaggtgttgccgccggcctgggcggcggtgctggtggcgcctctcggcggaacagttttgagattgatcggaggggtagttttgagattggctttgaggttgctccagtcgtgtccacaggtgggccctgtccctcacctaactgttcatctccctccagccattgcacgtgctgccgtatgttgcatacccgcttccaagtatcgagtgctgcgtgcccgttcaagtgggtttctagtgcttccgcgttctggttggtagcacagcagcctgcttgcaaggtgccacgtcggctctcaacacactaatgattcgctgcattactcttgcctctgccggtgtgacttcgcaggcctgcagcaggcgtcgggagcgcgcactggcgcggcacccgcccacgccgggggtggattcagctgcttgtctgcgcagctccacggcagcaggcagcacaagcagcagcagccggaagcggcagaacattccagcaagtag

SEQ ID NO: 4 - DNA, 23 bp, synthetic construct
  source 1..23: mol_type = other DNA; note = Encodes gRNA for target in exon 1 SEQ ID NO: 1
tgagcgccactagatcgtcgcgg

SEQ ID NO: 5 - DNA, 23 bp, synthetic construct
  source 1..23: mol_type = other DNA; note = Encodes gRNA for target in exon 4 SEQ ID NO: 1
ccgcttctatcgctctccagagg

SEQ ID NO: 6 - protein, 425 aa, Oocystis sp.
  source 1..425: note = PKL-0924 kinase-like protein mutated sequence
MRDPSGSATRPTPARVHLARDPSPCKPMPQDFSVSDQQGNGAFDAPDAAGAACTTLPRRSSGAHDCRDMVAQEHGDGAAQVAQPSQTSAVSKMTHELLATYQRCGGGVGPTQASDATHALHQLQQANQAPEQRQQAQRNLLTKPFKGLHNDGHDNENWDLIICVGDEFVSSSNLKYVVIDLLGQGTFGQVVRCWCDQTQEYVAVKVIKNQPAYYQQARVEVGLLQYLNRCADADDVRHIVRLRDYFLFRNHLCLAFELLSVNLYELIKHNQFRGLSAGLVRVFIAQLLDALVVLRESRLIHCDLKPENVLLTGAESADIKVIDFWFRLPGEQNGVQLHPEPLLSLSRGGAWLPVQRGDRHVVPGLHGRGAVPWPAAVPGRIGARFVVSGGAGGGPPAAVPAAGRKAHQQVFQDGGARGAAAKRQV

SEQ ID NO: 7 - protein, 81 aa, Oocystis sp.
  source 1..81: note = PKL-0924 kinase-like protein mutated sequence variant 30069
MRDPSGSATRPTPARVHLARDPSPCKPMPQDFSVSDQQGNGAFDAPDAAGAACTTLPRQGTATPRREIDGPATRNKAASLR

SEQ ID NO: 8 - DNA, 874 bp, Oocystis sp.
  source 1..874: mol_type = genomic DNA; note = PKL-0924 kinase-like protein core domain of wild-type genomic DNA sequence
ccgcgacgatctagtggcgctcacgactgcagggacatggtagcccaggaacacggagacggggccgcccaggtggcacagccatcccagacatcggctgtgtccagtagttcgtgtgtcatcttcgccacgtatcaacggtgcggtggaggcgttggaccgacacaagccagcgacgcaacgcacgcgttgcatcagctgcagcaggccaatcaagcaccggagcagcggcagcaggcacagcgcaatttgctcacgaagcctttcaagggtctgcataacgacggtcatgacaacgagaactgggacttgatcatatgtgtcggggacgagtttgtgtccagctccaacctcaagtacgtcgtcatcgacttgctgggccagggcacgttcggccaggtagtgcgctgctggtgcgaccagacgcaggagtatgtagccgtcaaggtgataaaaaatcagccggcttactatcagcaagcgcgcgtagaggttggcctgttgcaatacctcaaccgctgtgcggacgcggacgacgtccggcacattgtgcgcctccgcgactactttttgttccgtaaccacttgtgcctcgcgttcgagctgctgtcggtcaacctgtacgagctcatcaagcacaaccagttccgtgggctgtctgcggggctcgtgcgcgtgttcatcgctcagctgcttgatgcgctggtggtgctgcgtgagtctcgcctcatccactgcgacctcaagccggagaacgtgctgcttacgggcgctgagtcagctgacataaaggtcatcgactttgggtccgcttgcctggagagcaaaacggtgtacagctacatccagagccgcttctatcgctctccagagg

SEQ ID NO: 9 - protein, 331 aa, Oocystis sp.
  source 1..331: note = Core domain of PKL-0924 protein kinase-like domain
YVVIDLLGQGTFGQVVRCWCDQTQEYVAVKVIKNQPAYYQQARVEVGLLQYLNRCADADDVRHIVRLRDYFLFRNHLCLAFELLSVNLYELIKHNQFRGLSAGLVRVFIAQLLDALVVLRESRLIHCDLKPENVLLTGAESADIKVIDFGSACLESKTVYSYIQSRFYRSPEVVLGYPYNVAIDMWSLGCMAAELFLGLPLFPGASEHDLLSRVVQAVGLPPLYLLQGAKHTNKYFKMVERVVRLPSGRSEVVPEYVMRTAAEFEALTGLKATTGKRYFSHTRLQDIINSYPSEGAGSELRRSLLDFLRGVLDPDPAARWTPQQAARHPFV

(19) United States
(12) Patent Application Publication
(10) Pub. No.: US 2024/0150800 A1
(43) Pub. Date: May 9, 2024

(54) RECOMBINANT ALGAE HAVING HIGH BIOMASS AND LIPID PRODUCTIVITY

(71) Applicant: VIRIDOS, INC., La Jolla, CA (US)

(72) Inventors: Saheed Imam, La Jolla, CA (US); Eric Moellering, La Jolla, CA (US); Luke Peach, La Jolla, CA (US)

(21) Appl. No.: 18/386,502

(22) Filed: Nov. 2, 2023

Related U.S. Application Data
(60) Provisional application No. 63/422,815, filed on Nov. 4, 2022.

(51) Int. Cl. C12P 7/6436; C07K 14/405; C12N 1/12

(52) U.S. Cl. CPC C12P 7/6436; C07K 14/405; C12N 1/125; C12R 2001/89

(57) ABSTRACT

The invention provides a recombinant algal organism that has been genetically modified in a gene encoding a protein kinase-like protein. The recombinant organism exhibits higher biomass productivity and higher lipid productivity versus a corresponding control algal organism not having the genetic modification. The recombinant organism is therefore useful in applications requiring biomass and/or lipid productivity, e.g. in the production of biofuels or other lipidic matter. Methods of using the organism and biomass containing or produced by the organism are also provided.

### CROSS-REFERENCE TO RELATED APPLICATIONS

This application claims the benefit of priority under 35 U.S.C. § 119(e) of U.S. Provisional Application No. 63/422,815 filed Nov. 4, 2022. The disclosure of the prior application is considered part of and is herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name SGI2310-1_SL, was created on Oct. 31, 2023, and is 24 kb in size.

### FIELD OF THE INVENTION

The invention involves recombinant algae and methods for producing biomass and lipids.

### BACKGROUND OF THE INVENTION

The production of biofuels presents great opportunities to develop environmentally sound sources of energy that can be obtained at reasonable cost. Efforts have been directed towards using algae or other microorganisms to produce hydrocarbons that can be used as biodiesel or other biofuels due to their high lipid content. Additional specialty chemicals can also be obtained from these organisms and for use in consumer products.

Since algae use energy from sunlight to combine water and carbon dioxide to produce biomass, achieving increased productivity offers the possibility of a carbon neutral fuel source. The development of algal strains with very high lipid productivity for the production of algal-sourced biofuels therefore presents the possibility of a significant reduction in new carbon dioxide released into the atmosphere and a consequent reduction in the problem of global warming.

The development of commercially viable algal biofuels requires strains with high lipid and biomass productivity. Even the most productive wild type strains are not sufficiently productive to permit an economically viable development of this resource. Strategies for increasing algal production of biofuels and other products have included modification of nutrition provided to the organisms, such as cultivating the organisms in nitrogen, phosphorus, or silicon deficient media. Other strategies have included modification of cultivation conditions or environmental protocols, or various efforts directed towards genetic engineering of the organisms. While engineering algae strains to have a combination of increased photosynthetic efficiency (resulting in increased overall biomass productivity) and/or high lipid productivity could provide a solution to this problem, deficiencies still remain. The development of higher performing strains continues to be a barrier to efficient utilization of this energy source.

### SUMMARY OF THE INVENTION

The invention provides a recombinant algal organism that has been genetically modified in a gene encoding a protein kinase-like protein. The recombinant organism exhibits higher biomass productivity and higher lipid productivity versus a corresponding control algal organism not having the genetic modification. The recombinant organism is therefore useful in applications requiring biomass and/or lipid productivity, e.g. in the production of biofuels or other lipidic matter. Methods of using the organism and biomass containing or produced by the organism is also provided.

In a first aspect the invention provides a recombinant algal organism having a deletion, disruption, or inactivation of a gene encoding a protein kinase-like domain having a polypeptide sequence having at least 80% sequence identity to SEQ ID NO: 1; or a gene encoding a protein kinase-like domain having a nucleic acid sequence having at least 80% sequence identity to SEQ ID NO: 2 or 3, where the recombinant algal organism exhibits higher biomass productivity and higher lipid productivity versus a corresponding control algal organism not having the deletion, disruption, or inactivation. In one embodiment the recombinant algal organism can have a gene encoding a protein kinase-like domain having a polypeptide sequence having at least 85% sequence identity to SEQ ID NO: 1. In another embodiment the recombinant algal organism can have gene encoding a protein kinase-like domain having a nucleic acid sequence having at least 85% sequence identity to SEQ ID NO: 3. In various embodiments the recombinant algal organism is a Chlorophyte alga, which can be an organism is of the Class Trebouxiophyceae. The deletion, disruption, or inactivation can be to a regulatory sequence of the gene encoding the protein kinase-like domain. In any embodiment the regulatory sequence can be a promoter. In any embodiment the deletion, disruption, or inactivation can be a deletion of one or more amino acids of the encoded protein kinase-like domain, or can be an insertion in the gene encoding the kinase-like protein. The insertion can involve insertion of a stop codon in a sequence encoding the kinase-like domain. In any embodiment the recombinant algal organism can have at least 20% or 25% higher lipid productivity versus a control algae. In any embodiment the recombinant algal organism can have an at least 35% higher biomass productivity per unit time versus the corresponding control algal cell or organism. In any embodiment the recombinant algal organism can have a FAME/TOC ratio of at least 0.4 after two days of cultivation. In any embodiment the recombinant algal organism can have higher biomass productivity and/or higher total organic carbon production under nitrogen deficient conditions. In various embodiments the recombinant algal organism can be from a family selected from the group consisting of: Oocystaceae, Chlorellaceae, and Eustigmatophyceae. In any embodiment the recombinant algal organism can be from a genus selected from the group consisting of: Chlorella, Parachlorella, Picochlorum, Tetraselmis, and Oocystis.

In some embodiments the protein kinase-like domain can have a polypeptide sequence having at least 90% sequence identity to SEQ ID NO: 1. In some embodiment the gene encoding the protein kinase-like domain can have at least 90% sequence identity to SEQ ID NO: 2 or SEQ ID NO: 3.

In another aspect the invention provides a biomass product containing any recombinant alga described herein.

In another aspect the invention provides a recombinant algal organism having a deletion, disruption, or inactivation of a gene encoding a protein kinase-like domain having a polypeptide sequence having at least 90% sequence identity to SEQ ID NO: 8, where the recombinant algal organism exhibits higher biomass productivity and higher lipid productivity versus a corresponding control algal organism not having the genetic modification. In any embodiment the genetic modification can be a deletion, disruption, or inactivation disclosed herein.

In another aspect the invention provides a method of producing a composition containing lipids. The methods involve performing a genetic modification to an algal organism in an algal organism to a gene encoding a protein kinase-like domain having a polypeptide sequence having at least 80% sequence identity to SEQ ID NO: 1; or to a gene encoding a protein kinase-like domain having a nucleic acid sequence having at least 80% sequence identity to SEQ ID NO: 2 or 3, where the recombinant algal organism exhibits higher biomass productivity and higher lipid productivity versus a corresponding control algal organism not having the genetic modification, and thereby produce a composition containing lipids. The methods can also involve a step of cultivating the organism.

In any embodiment the method can involve a step of harvesting a lipidic composition from the algal organism. The genetic modifications to the sequence encoding the protein-kinase like domain can be a deletion, disruption, or inactivation. The recombinant alga can have at least 50% greater lipid productivity versus a control alga.

In another aspect the invention provides a method of producing a composition containing lipids. The method involves performing a genetic modification to an algal organism described herein, cultivating the organism, and thereby producing a composition containing lipids. In some embodiments the methods include a step of harvesting a lipidic composition from the algal organism.

### DETAILED DESCRIPTION OF THE DRAWINGS

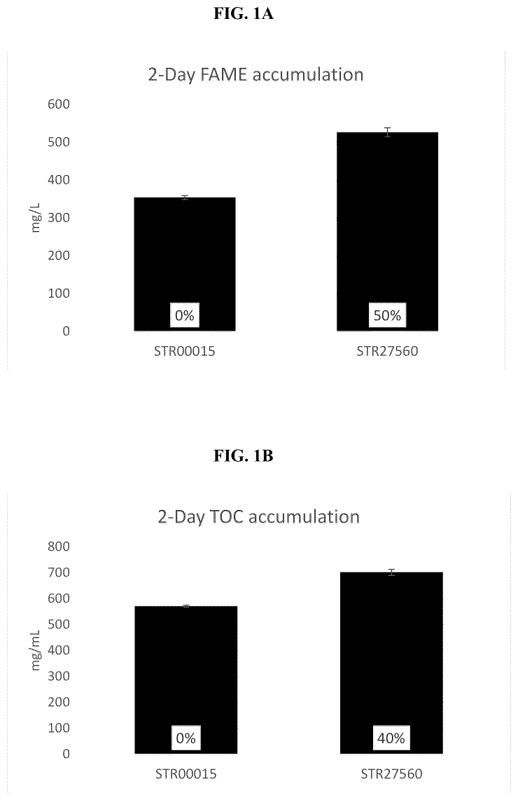

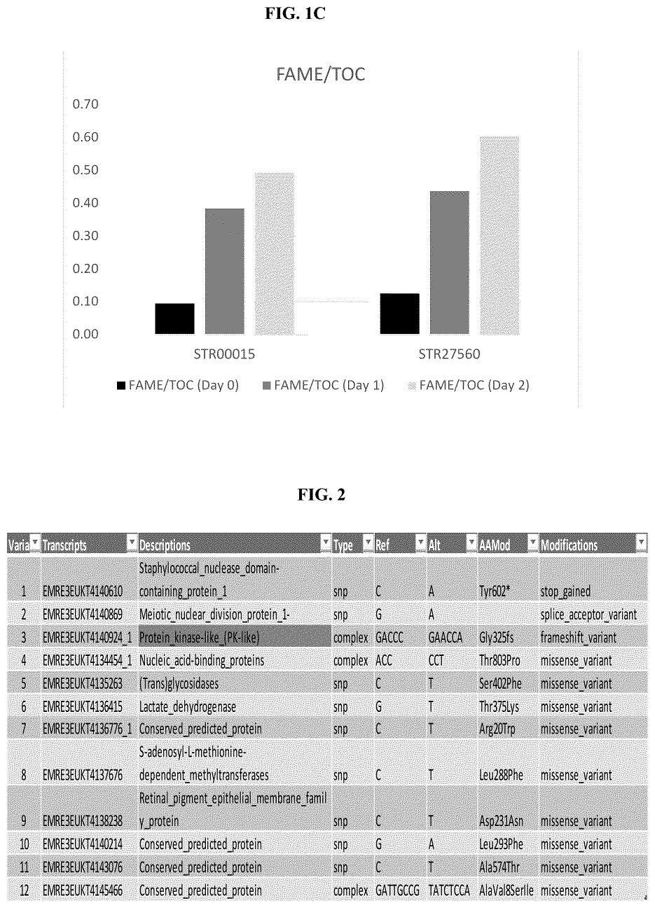

FIGS. 1A-1C. FIG. 1A provides a bar graph demonstrating an increase in 2-day FAME accumulation for the mutated strain (STR27560) versus the parent wild-type strain (STR00015). FIG. 1B provides a bar graph demonstrating an increase in 2-day TOC accumulation for the mutated strain versus the parent wild-type strain. FIG. 1C provides a bar graph demonstrating that the mutated strain has a higher FAME/TOC ratio than the parental wild-type strain.

FIG. 2 provides a table of the list of mutations identified within exons or at splice junctions in the mutated strain STR27560.

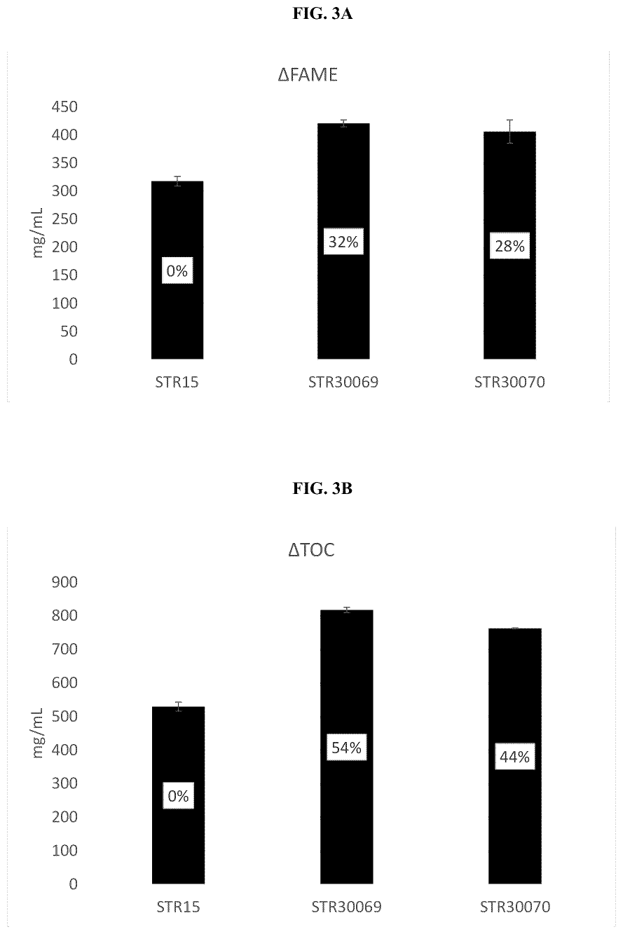

FIGS. 3A-3B. FIG. 3A provides a bar graph demonstrating higher FAME production in two wild-type parent strains recapitulated with a disruption of the protein-kinase like domain from FIG. 2 (STR30069 and STR30070). FIG. 3B provides a bar graph showing an increase in TOC accumulation in two wild-type parent strains recapitulated with a disruption of the protein-kinase like domain from FIG. 2.

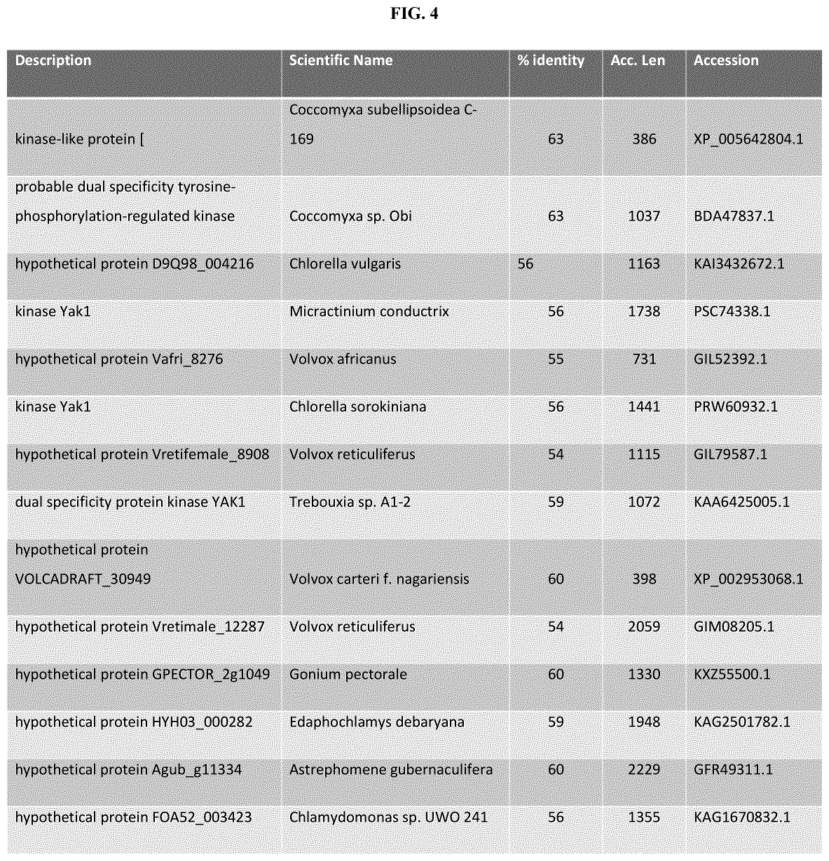

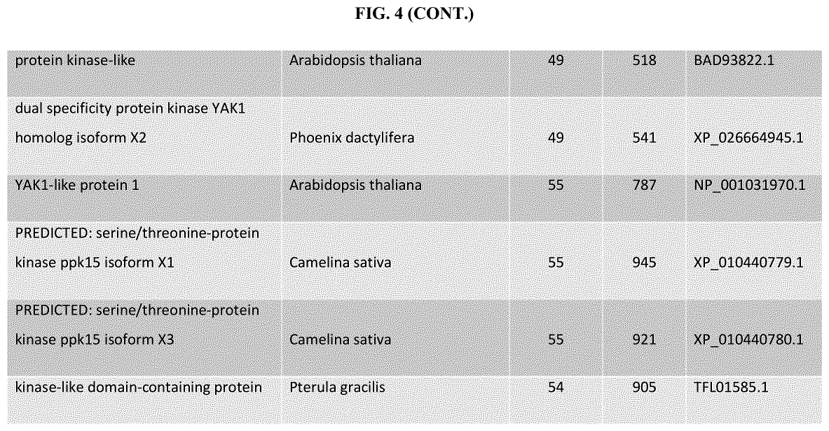

FIG. 4 provides a table of orthologs of PKL-0924 (the gene encoding the “kinase-like domain”) in various green algae.

### DETAILED DESCRIPTION OF THE INVENTION

The invention provides a recombinant algal organism that has been genetically modified in a gene encoding a protein kinase-like domain. The recombinant organism exhibits higher biomass productivity and higher lipid productivity versus a corresponding control algal organism not having the genetic modification. The recombinant organism is therefore useful in applications requiring biomass and/or lipid productivity. It was surprisingly discovered that this single gene modification results in a strain having significantly higher lipid and biomass productivity. In any embodiment the genetic modification can be a deletion, disruption, or inactivation of a gene encoding a protein kinase-like domain.

The recombinant cell or organism of the invention having a genetic modification described herein can have higher lipid productivity (e.g. as measured by FAME) and/or higher biomass productivity versus a corresponding (control) cell or organism. In some embodiments the genetic modification is an attenuation(s) of a gene encoding a kinase-like domain. In any embodiment biomass productivity can be measured as the rate of biomass accumulation, for example as measured by the total organic carbon (TOC) content of the respective cells or organisms.

In one embodiment the lipid and/or biomass productivity is higher in batch culture, i.e. a culture where nutrients are not renewed or re-supplied to the medium during culturing, compared to a corresponding (control) cell or organism. Any of the mutant cells or organisms disclosed herein can be photosynthetic cells or organisms. Any of the recombinant (mutant) cells or organisms described herein can exhibit increased lipid productivity and/or increased biomass productivity under photoautotrophic conditions compared to a corresponding control cell or organism, i.e. conditions where the recombinant cells or organisms can produce their own biomass using light, carbon dioxide, water, and nutrients via photosynthesis. Corresponding (control) cells or organisms are cells or organisms that are useful for evaluating the effect of any one or more of the genetic modifications. Corresponding (“control”) cells or organisms are cells or organisms that do not have the one or more genetic modifications being evaluated and that are subjected to the same or substantially the same conditions as the test cells or organisms such that a difference in the performance or characteristics of the cells or organisms is based only on the genetic modification(s) being evaluated. In any embodiment the corresponding (control) cells or organisms can be of the same species as the test organism. They can also be the same or similar in every way except for the genetic modification(s) being evaluated. In some embodiments the corresponding (control) cell or organism is a wild-type cell or organism. But the corresponding (control) cell or organism can also be a laboratory strain or parental strain of the test cell or organism. Substantially the same conditions can be the same conditions or slightly different conditions where the difference does not materially affect the function, activity, or expression of the nucleic acid sequence modified.

In any embodiment the recombinant cells or organisms can be algal cells. In one embodiment the recombinant alga has a genetic modification to a gene encoding a protein kinase-like domain. The lipid products of these mutants can be further processed into biofuels or used in the production of other specialty chemical products.

The genes encoding the protein kinase-like domain can be any of the nucleic acid sequences described herein, which can encode a protein kinase-like domain. In some embodiments the encoded the protein kinase-like domain can have a polypeptide sequence of SEQ ID NO: 1 or 9, or a polypeptide sequence having at least 80%, or at least 85%, or at least 90%, or at least 95%, or at least 98% sequence identity to SEQ ID NO: 1 or 9, which in any embodiment can be a sequence of at least 100, or at least 200, or at least 300, or at least 500, or at least 600, or at least 700, or at least 800, or at least 1000 amino acids; and/or the gene encoding the protein kinase-like domain can have a sequence selected from SEQ ID NOs: 2 or 3, or a sequence having at least 80%, or at least 85%, or at least 90%, or at least 95%, or at least 98% sequence identity to SEQ ID NO: 2 or 3, which in any embodiment can be a sequence of at least 100, or at least 200, or at least 300, or at least 500, or at least 600, or at least 700, or at least 1000 nucleotides. In some embodiments the sequence encoding the protein kinase-like domain can have any of the nucleic acid sequences described herein, and can encode any of the polypeptide sequences disclosed herein; the nucleic acid and polypeptide sequences are hereby disclosed in all possible combinations and sub-combinations.

In some embodiments recombinant cells or organisms of the invention can have a reduced amount of chlorophyll b, and can have an increased chlorophyll a to chlorophyll b ratio (chl a/chl b) compared to a corresponding control cell or organism. The recombinant cells or organisms can have decreased photosynthetic antenna size, for example reduced photosystem II (PSII) and/or reduced photosystem I (PSI) antenna size. In various embodiments the cross-sectional unit size of the PSII and/or PSI antenna of the recombinant cells or organisms disclosed herein can be reduced by at least 10%, at least 20%, at least 30%, at least 25%, at least 30%, at least 35%, at least 40%, at least 45%, at least 50%, or at least 60% compared to the PSII and/or PSI antenna size of a corresponding control cell or organism.

As used herein, “exogenous” with respect to a nucleic acid or gene indicates that the nucleic acid or gene has been introduced (e.g. “transformed”) into an organism or cell by human intervention. For example, such an exogenous nucleic acid can be introduced into a cell or organism via a recombinant nucleic acid construct. An exogenous nucleic acid can be a sequence from one species introduced into another species, i.e., a “heterologous” nucleic acid. A heterologous nucleic acid can also be an exogenous synthetic sequence not found in the species into which it is introduced. An exogenous nucleic acid can also be a sequence that is homologous to an organism (i.e., the nucleic acid sequence occurs naturally in that species or encodes a polypeptide that occurs naturally in the host species) that has been isolated and subsequently reintroduced into cells of that organism. In some embodiments an exogenous nucleic acid that includes a homologous sequence can be distinguished from the naturally-occurring sequence by the presence of non-natural sequences linked to the exogenous nucleic acid, which can include but are not limited to non-native regulatory sequences attached to the homologous gene sequence in a recombinant nucleic acid construct. Alternatively or in addition, a stably transformed exogenous nucleic acid can be detected and/or distinguished from a native gene by its juxtaposition to sequences in the genome where it has integrated. Further, a nucleic acid is considered exogenous if it has been introduced into a progenitor of the cell, organism, or strain under consideration.

A “recombinant” or “engineered” nucleic acid molecule is a nucleic acid molecule that has been altered through human manipulation. As non-limiting examples, a recombinant nucleic acid molecule includes any nucleic acid molecule that: 1) has been partially or fully synthesized or modified in vitro, for example, using chemical or enzymatic techniques (e.g., by use of chemical nucleic acid synthesis, or by use of enzymes for the replication, polymerization, digestion (exonucleolytic or endonucleolytic), ligation, reverse transcription, transcription, base modification (including, e.g., methylation), integration or recombination (including homologous and site-specific recombination) of nucleic acid molecules); 2) includes conjoined nucleotide sequences that are not conjoined in Nature; 3) has been engineered using molecular biology techniques such that it lacks one or more nucleotides with respect to the naturally occurring nucleic acid molecule sequence; and/or 4) has been manipulated using molecular biology techniques such that it has one or more sequence changes or rearrangements with respect to the naturally occurring nucleic acid sequence, or has a sequence (e.g. by insertion) not found in the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

When applied to organisms, the terms “transgenic” “transformed” or “recombinant” or “engineered” or “genetically engineered” refer to organisms that have been manipulated by introduction of an exogenous or recombinant nucleic acid sequence into the organism, or by genetic modification of native sequences (which are therefore then recombinant). Recombinant or genetically engineered organisms can also be organisms into which constructs for gene “knock down,” deletion, attenuation, inactivation, or disruption have been introduced to perform the indicated manipulation. Such constructs include, but are not limited to, RNAi, microRNA, shRNA, anti sense, and ribozyme constructs. In any embodiment the constructs can be exogenous and/or introduced by human activity. A recombinant organism can also include those having an introduced exogenous regulatory sequence operably linked to an endogenous gene of the transgenic microorganism, which can enable transcription in the organism. Also included are organisms whose genomes have been altered by the activity of meganucleases or zinc finger nucleases. A heterologous or recombinant nucleic acid molecule can be integrated into a genetically engineered/recombinant organism's genome or, in other instances, not integrated into a recombinant/genetically engineered organism's genome, or can be present on a vector or other nucleic acid construct. As used herein, “recombinant microorganism” or “recombinant host cell” includes progeny or derivatives of the recombinant microorganisms of the disclosure.

Any of the recombinant algal cells or organisms described herein can be generated by human action, for example, by classical mutagenesis and/or genetic engineering, but can also be produced by any feasible mutagenesis method, including but not limited to exposure to UV light, CRISPR/Cas9, cre/lox, gamma irradiation, or chemical mutagenesis. Screening methods can be used to identify mutants having desirable characteristics (e.g., reduced chlorophyll and increased lipid and/or biomass productivity. Methods for generating mutants of algal organisms using classical mutagenesis, genetic engineering, and phenotype or genotype screening are well-known in the art.

### Algal Cell or Organism

The recombinant algal cell or organism of the invention can be a mutant microalga, or a mutant photosynthetic organism, or a mutant green alga. The recombinant alga can be any eukaryotic microoalga such as, but not limited to, a Chlorophyte, an Ochrophyte, or a Charophyte alga. In some embodiments the mutant microalga can be a Chlorophyte alga of the taxonomic Class Chlorophyceace, or of the Class Chlorodendrophyceae, or the Class Prasinophyceace, or the Class Trebouxiophyceae, or the Class Eustigmatophyceae. In some embodiments, the mutant microalga can be a member of the Class Chlorophyceace, such as a species of any one or more of the genera Asteromonas, Ankistrodesmus, Carteria, Chlamydomonas, Chlorococcum, Chlorogonium, Chrysosphaera, Dunaliella, Haematococcus, Monoraphidium, Neochloris, Oedogonium, Pelagomonas, Pleurococcus, Pyrobotrys, Scenedesmus, or Volvox. In other embodiments the mutant microalga of the invention can be a member of the Order Chlorodendrales, or Chlorellales. In other embodiments, the mutant microalga can be a member of the Class Chlorodendrophyceae, such as a species of any one or more of the genera Prasinocladus, Scherffelia, or Tetraselmis. In further alternative embodiments, the mutant alga can be a member of the Class Prasinophyceace, optionally a species of any one or more of the genera Ostreococcus or Micromonas. Further alternatively, the mutant microalga can be a member of the Class Trebouxiophyceae, and optionally of the Order Chlorellales, and optionally a genera selected from any one or more of Botryococcus, Chlorella, Auxenochlorella, Heveochlorella, Marinichlorella, Oocystis, Parachlorella, Pseudochlorella, Tetrachlorella, Eremosphaera, Franceia, Micractinium, Nannochloris, Picochlorum, Prototheca, Stichococcus, or Viridiella, or any of all possible combinations or sub-combination of the genera. In another embodiment the recombinant alga can be a Chlorophyte alga of the Class Trebouxiophyceae and the family Coccomyxaceae, and the genus Coccomyxa (e.g. Coccomyxa subellipsoidea). Or of the family Chlamydomonadaceae and the genus Chlamydomonas (e.g. Chlamydomonas reinhardtii); or of the family Volvocaceae and the genus Volvox (e.g. Volvox carteri, Volvox aureus, Volvox globator).

In another embodiment the recombinant alga is a Chlorophyte alga of the Class Trebouxiophyceae, or Eustigmatophyceae, and can be of the Order Chlorellales or Chlorodendrales, and can be of the Family Oocystaceae, or Chlorellaceae, or Monodopsidaceae, and optionally from a genus selected from one or more of Oocystis, Parachlorella, Picochlorum, Nannochloropsis, and Tetraselmis. The recombinant alga can also be from the genus Oocystis, or the genus Parachlorella, or the genus Picochlorum, or the genus Tetraselmis, or from any of all possible combinations and sub-combinations of the genera. In one embodiment the recombinant algal cell or organism is of the Class Trebouxiophyceae, of the Order Chlorellales, and optionally of the family Oocystaceae, and optionally can be of the genus Oocystis.

### Genetic Modification

A “genetic modification” can be any one or more of a mutation, a disruption or gene “knock out,” a deletion, an insertion, insertion of a stop codon, an inactivation, an attenuation, a rearrangement, one or more point mutations, a frameshift mutation, an inversion, a single nucleotide polymorphism (SNP), a truncation, a point mutation, that changes the activity or expression of the one or more gene or nucleic acids. In some embodiments the change in expression is a reduction in expression or an elimination of the expression or activity. The genetic modification can be made or be present in any sequence that affects expression or activity of the gene or nucleic acid sequence, or the nature or quantity of its product, for example to a coding or non-coding sequence, a promoter, a terminator, an exon, an intron, a 3′ or 5′ UTR, or other regulatory sequence; a genetic modification performed in any structure of the gene can result in attenuation or elimination of the gene or nucleic acid product or activity. In one embodiment the genetic modification is a deletion, disruption, or inactivation. The genetic modification can be made to or be present in the host cell's native genome. In some embodiments, a recombinant cell or organism having attenuated expression of a gene as disclosed herein can have one or more mutations, which can be one or more nucleobase changes and/or one or more nucleobase deletions and/or one or more nucleobase insertions, into the region of a gene 5′ of the transcriptional start site, such as, in non-limiting examples, within about 2 kb, within about 1.5 kb, within about 1 kb, or within about 0.5 kb of the known or putative transcriptional start site, or within about 3 kb, within about 2.5 kb, within about 2 kb, within about 1.5 kb, within about 1 kb, or within about 0.5 kb of the translational start site.

An “attenuation” is a genetic modification resulting in a reduction of the function, activity, or expression of a gene or nucleic acid sequence compared to a corresponding (control) cell or organism not having the genetic modification being examined, i.e. the diminished function, activity, or expression is due to the genetic modification. The activity of a nucleic acid sequence can be expression of an encoded product, a binding activity (e.g. RNA binding), or other activity the nucleic acid sequence exerts within the organism. In various embodiments an attenuated gene or nucleic acid sequence produces less than 90%, or less than 80%, or less than 70%, or less than 50%, or less than 30%, or less than 20%, or less than 10%, or less than 5% or less than 1% of its function, activity, or expression of the gene or nucleic acid sequence compared to the corresponding (control) cell or organism. In various embodiments a gene attenuation can be achieved via a deletion, a disruption, or an inactivation. Any of the genetic modifications described herein can result in partial or complete attenuation of the function, activity, or expression of the attenuated gene or nucleic acid sequence.

An unmodified gene or nucleic acid sequence present naturally in the organism denotes a natural, endogenous, or wild type sequence. A deletion can mean that at least part of the object nucleic acid sequence is deleted, or that the entire sequence is deleted.

A disruption (or “knock out”) is a genetic modification that removes at least so much of the function, activity, or expression of a gene or nucleic acid sequence that the function, activity, or expression of the gene or nucleic acid sequence has no significant effect on the cell or organism compared to a corresponding (control) cell or organism not having the disruption and cultivated under the same or substantially the same conditions. A deletion, disruption or “knock out”, or inactivation can also remove all function, activity, or expression of a gene or nucleic acid sequence. A “disruption” (or “knock out”) of a gene can be performed in various ways, e.g. by insertion or deletion of a nucleotide sequence into or from the coding, non-coding, or regulatory portion of a gene with resulting loss of function, activity, or expression of the gene; the loss can be such that the function, activity, or expression of the gene has no significant effect on lipid or biomass productivity. In one embodiment a disruption can be performed by insertion of a stop codon (or other disrupting sequence) into the coding, non-coding, or regulatory portion of the gene. A disruption can also be performed by effecting a single or multi-nucleotide polymorphism into the coding, non-coding, or regulatory portion of the gene, which can result in transcription of an inactive or non-functional protein. An “inactivation” is a type of functional deletion causing loss of activity or expression of an inactivated gene or nucleic acid sequence. An “inactivation” can be reversible or irreversible (for example the reversible or irreversible binding of a component to the gene or nucleic acid sequence). Thus, deletions, disruptions, and inactivations can also be attenuations. An attenuation can also be a downregulation of a gene or nucleic acid sequence, which refers to the cell or organism decreasing the amount of function, activity, or expression. Functional expression refers to the expression of a functional product or activity of a nucleic acid sequence. When the expressed product of a nucleic acid is a polypeptide, functional expression means expression of polypeptide activity having at least 10% or at least 25% or at least 50% or at least 75% of the activity of an unmodified cell or organism. For activity of a gene or nucleic acid sequence functional expression means activity or expression of at least 10% or at least 25% or at least 50% or at least 75% of the activity or expression of a corresponding (control) cell or organism not having the modification and cultivated under the same or substantially the same conditions. Thus, various types of genetic modifications can be given terms that overlap in description. Persons of ordinary skill know that the particular term describing a genetic modification can be dependent both on how a gene or its components, or nucleic acid sequence is being physically changed as well as on the context. The recombinant cells or organisms of the invention can have any of the types of genetic modifications described herein.

In one embodiment the genetic modification is a disruption (or “knock out”) involving the introduction of a stop codon into a gene (including regulatory sequences, e.g. a promoter), or nucleic acid sequence encoding a protein kinase-like domain described herein. In one embodiment the genetic modification can be a stop mutation introduced into SEQ ID NOs: 2-3 or 8 (coding sequence and genomic DNA sequence of protein kinase-like domain from Oocystis sp.) or into a variant of either, or into a nucleic acid sequence encoding the polypeptide of SEQ ID NO: 1 or 9 (protein kinase-like domain polypeptide sequence in Oocystis sp.), or into a variant thereof, or into a regulatory sequence of any of SEQ ID NO: 2-3 or 8, or a gene encoding the polypeptide sequence of SEQ ID NO: 1 or 9.

Variant sequences have at least 60% sequence identity or at least 70% sequence identity or at least 75% or at least 80% or at least 85% or at least 90% or at least 95% or at least 98% sequence identity to any nucleotide or polypeptide sequence to the reference sequence, which can be any of SEQ ID NOs: 1-3 or 8-9.

In other embodiments the genetic modification can also be a stop mutation or nonsense mutation introduced into a gene or nucleic acid sequence encoding a protein kinase-like domain disclosed herein. In various embodiments the gene or nucleic acid sequence is SEQ ID NO: 2-3 or 8 (or a variant of any) or a gene or nucleic acid sequence encoding the polypeptide of SEQ ID NO: 1 or 9, or a variant thereof. The stop mutation can be introduced at any location of the sequence or into a regulatory sequence governing the sequence, where the modification results in a termination of transcription from the gene prior to its natural point. Thus, in one embodiment the mutation is the introduction of a stop codon that functionally deletes or disrupts the activity or expression of the gene or nucleic acid sequence. The stop codon or other modification can also be introduced at many different loci or locations within a gene encoding a protein kinase-like domain, or in a regulatory sequence, for example at a promoter, terminator, or other regulatory sequence that attenuates the gene or the activity of the encoded polypeptide, and that results in functional deletion of the gene. Analogous modifications can be made to the sequence(s) for similar effect. Such insertion or deletion or other modification can also cause a loss of function or activity in the protein kinase-like protein and result in the effect of increased lipid productivity and/or increase biomass productivity.

Any of the recombinant cells or organisms of the invention can have a reduced functional absorption cross section of PSII and/or reduced PSII antenna size. For example, the cross-sectional unit size of the PSII antenna can be reduced by at least about 10%, at least 20%, at least 30%, at least 25%, at least 30%, at least 35%, at least 40%, at least 45%, at least 50%, at least 60%, at least about 70%, or at least about 80% compared to the functional absorption cross section of PSII and/or PSII antenna size of the corresponding (control) cell or organism not having the genetic modification. In some embodiments the recombinant cells or organisms of the invention can additionally (and optionally) have a reduced functional absorption cross section of PSI or reduced PSI antenna size by the same amounts stated above versus a corresponding (control) cell or organism.

In some embodiments, a recombinant algal cell or organism as provided herein can have increased Fv/Fm with respect to a corresponding control cell or organism. For example, the mutant photosynthetic organism may have Fv/Fm increased by at least 5%, at least 10%, at least 12%, at least 15%, at least 20%, at least 30%, at least 40% or at least 50% compared to a corresponding (control) photosynthetic organism. In various embodiments the Fv/Fm can be increased by 5-50%, or by 5-30% or by 5-20% with respect to a control photosynthetic organism.

Further, a mutant photosynthetic organism as provided herein can have an increased rate of electron transport on the acceptor side of photosystem II with respect to a control or wild type cell. The rate can be at least about 20%, 30%, 40%, 50%, 60%, 80%, or 100% higher compared to a corresponding control cell or organism. In addition, mutant photosynthetic cells or organisms of the invention can have a rate of carbon fixation (Pmax (C)) in a recombinant cell or organism as provided herein can be elevated with respect to a control organism. For example, Pmax (14C) can be increased by at least about 20%, 30%, 40%, 50%, 60%, 80%, or 100% compared to a corresponding control cell or organism.

In some embodiments, the recombinant cells or organisms of the invention have decreased PSI and/or PRI antenna size and can optionally also have a higher amount of a ribulose bisphosphate carboxylase activase (Rubisco activase or “RA”) than a corresponding (control) or wild type organism, for example, at least 1.2, 1.4, 1.6, 1.8, 2, 2.2, or 2.5 fold the amount of RA as a control organism. In some embodiments, the mutants demonstrate reduced expression of 6, 8, 10, 12, or 14 LHCP genes and increased expression of an RA gene, such as an RA-a or RA-P gene. Thus, the recombinant cells or organisms of the invention can be mutant photosynthetic organisms having reduced chlorophyll and reduced PRI antenna size where the mutants have a higher amount of Rubisco activase than control photosynthetic organisms.

The LHC super-gene family encodes the light-harvesting chlorophyll a/b-binding (LHC) proteins that constitute the antenna system of the photosynthetic apparatus. A recombinant algal mutant of the invention can also have a reduced expression of one or more LHC genes. Thus, in some embodiments the recombinant cells or organisms of the invention have at least 6, at least 8, at least 10, or at least 12 LHC genes that are attenuated or downregulated with respect to their expression level in a corresponding (control) cell or organism. In various embodiments the reduction in expression of the one or more LHC genes can be a reduction of at least 20%, or at least 30%, or at least 40%, or at least 50%, or at least 60%, or at least 70% in the level of LHC transcripts compared to the control cell or organism.

The structure of a gene consists of many elements, of which the protein coding sequence is only one part. The gene includes nucleic acid sequences that are not transcribed and sequences that are untranslated regions of the RNA. Genes also contain regulatory sequences, which includes promoters, terminators, enhancers, silencers, introns, 3′ and 5′ UTRs, and coding sequences, as well as other sequences known to be a part of genes. In various embodiments any of these structures or nucleic acid sequences can have one or more of the genetic modifications described herein that result in the higher lipid productivity and/or higher biomass productivity as described herein.

The recombinant cells or organisms can have a higher growth rate and/or a higher biomass productivity or lipid productivity than a corresponding control cell or organism not having the genetic modification, for example, higher biomass or lipid productivity per hour or per day or per period of 2 days or 3 days or 4 days or 5 days or 6 days. “Biomass” refers to cellular mass, whether of living or dead cells. Biomass productivity, or biomass accumulation, or growth rate, can be measured by any means accepted in the art, for example as ash free dry weight (AFDW), dry weight, wet weight, or total organic carbon (TOC) productivity. In any embodiment biomass productivity, or biomass accumulation, or the growth rate, can be measured as total organic carbon (TOC) productivity.

The recombinant cells or organisms of the invention can produce a greater amount of a biomass or lipid per time period (e.g. per minute or per hour or per day or per period of 2 days or 3 days or 4 days or 5 days or 6 days), for example the biomass can be a lipid product (which can optionally be measured as FAME), a carbohydrate, a protein product, a polyketide, a terpenoid, a pigment, an antioxidant, a vitamin, one or more nucleotides, one or more nucleic acids, one or more amino acids, one or more carbohydrates, an alcohol, a hormone, a cytokine, a peptide, or a polymer than a corresponding (control) organism not having the genetic modification(s). The amount of product can be expressed as g/time period, mg/time period, ug/time period, or any other defined quantity per defined time period described herein. Such bioproducts can be isolated from a lysate or biomass or cellular secretion of any of the recombinant cells or organisms of the invention. In some embodiments, the recombinant cells or organisms of the invention produce at least 10%, at least 15%, at least 20%, at least 25%, at least 30%, at least 35%, at least 40%, at least 45%, at least 50%, at least 60%, at least 70%, at least 80%, at least 90% or at least 100% or at least 200% more of a lipid or other bioproduct than a corresponding control alga cultured under the substantially the same conditions, which can be batch, semi-continuous, or continuous culture conditions and may be nutrient replete culture conditions or may be nitrogen deplete/deficient conditions, and may be photoautotrophic conditions. Continuous culture refers to a culture of continuous nutrient replenishment, and semi-continuous culture refers to nutrient replenishment once per day, both of which are through removal of culture and resupply.

### Increased Lipid Productivity

The recombinant mutant algae of the invention having a genetic modification to a gene or nucleic acid sequence encoding a protein kinase-like domain as described herein can demonstrate an increase in the production of lipid in the cell or organism versus a corresponding (control) cell or organism. The increase in lipid production can be measured by any accepted and suitable method, for example using fatty acid methyl ester (FAME) analysis. In one embodiment the increase in lipid production is measured as an increase in total FAME produced by the recombinant organisms. The recombinant cells or organisms of the invention having a genetic modification to a gene or nucleic acid encoding a protein kinase-like domain can exhibit at least 15% or at least 20% or at least 30% or at least 40% or at least 50% or at least 60% or at least 70% or at least 80% or at least 90% or at least 100% higher lipid productivity compared to a corresponding control cell or organism, as described herein. In other embodiments the increase in lipid productivity can be 15-25% or 15-35% or 15-45% or 15-50% or 25-45% or 25-55% or 25-70% or 25-90% or 25-100% or 25-150% or 25-200% or 30-35% or 30-45% or 30-55%. The increase can be weight for weight (w/w). In one embodiment lipid productivity is measured using the FAME profile (fatty acid methyl ester assay) of the respective cells or organisms. In one embodiment lipid productivity can be expressed as mg/L. In other embodiments the recombinant cells or organisms of the invention can exhibit at least 50 g/m2 or at least 60 or at least 70 or at least 80 grams per square meter of FAME accumulation after 5 days of cultivation. Methods of producing a FAME profile are known to persons of ordinary skill in the art. A FAME profile can be determined using any suitable and accepted method, for example a method accepted by most persons of ordinary skill in the art. The recombinant cell or organisms of the invention can, optionally, also have an increase in biomass productivity can be 15-35% or 15-40% or 25-45% or 15-50% or 25-70% or 50-100% or 50-200% (w/w).

An increase in lipid production or lipid productivity can be measured by weight, but can also be measured in grams per square meter per day of the surface of a cultivation vessel (e.g. a flask, photobioreactor, cultivation pond). In various embodiments the recombinant alga of the invention produce at least 3 or at least 4 or at least 5 or at least 6 or at least 7 or at least 8 or at least 10 or at least 12 or at least 13 or at least 14 grams per square meter per day of lipid production, which can be measured by the FAME profile. In any of the embodiments the high lipid and/or high biomass productivity phenotype can be obtained under nitrogen deplete conditions, which in some embodiments can involve dilution and/or replacement of medium with fresh nitrogen deplete medium during growth. Dilutions can be by any suitable amount, for example dilution by about 50% or by about 60% or by about 70% or at least 70%, or by about 80%, or by more than 80%. In one embodiment the lipid product is a fatty acid and/or derivative of a fatty acid. In one embodiment the fatty acids and/or derivatives of fatty acid comprise one or more species of molecules having a carbon chain between C8-C18 and/or C8-C20 and/or C8-C22 and/or C8-C24, in all possible combinations and sub-combinations. In one embodiment the growth conditions can be batch growth, involving spinning cells to remove nitrogen from the medium, replacing with nitrogen deplete medium, and resuming batch growth.

In any of the embodiments the genetic modification to the gene or nucleic acid sequence encoding the kinase-like domain can result in an attenuation of expression of the respective genes. The genetic modification can be any of those described herein. In one embodiment the genetic modification is a deletion, disruption, or inactivation. In another embodiment the genetic modification is a disruption of the gene.

### Biomass Productivity

The recombinant algal cells of the invention having a genetic modification to a gene or nucleic acid encoding a protein kinase-like domain described herein can also have higher biomass productivity than a corresponding (control) organism not having the genetic modification. Biomass can be measured using the total organic carbon (TOC) analysis, known to persons of ordinary skill in the art. The recombinant cells can have at least 20% higher or at least 25% higher or at least 30% higher or at least 35% higher, or at least 50% higher or at least 60% higher or at least 70% higher or at least 80% higher or at least 90% higher or at least 100% higher or at least 125% higher or at least 150% higher or at least 200% higher biomass productivity than a corresponding (control) cell or organism, which in one embodiment can be measured by total organic carbon analysis. In other embodiments the biomass productivity can be 15-35% or 15-40% or 25-45% or 15-50% or 25-70% or 50-100% or 50-200%.

Various methods of measuring total organic carbon are known to persons of ordinary skill in the art. Biomass productivity can be measured as mg/ml of culture per time period (e.g. 1 day or 2 days or 3 days or 4 days or 5 days). In some embodiments the higher biomass productivity and/or higher lipid productivity as described herein can occur under nitrogen deplete conditions. Thus, in one embodiment the recombinant alga of the invention can have higher lipid production and/or higher total organic carbon production than a corresponding (control) cell or organism, which higher amount can be produced under nitrogen deplete or low nitrogen conditions. Nitrogen deplete conditions can involve culturing in a buffer having less than 0.5 mM of nitrogen in any available form external to the cell or organism. In one embodiment the cells can be cultured in 0.5 mM or less of KNO3 or urea as a nitrogen source. Other buffers may also be used and be nitrogen deplete if they contain a level of nitrogen that does not change the physiology of a nitrogen-related parameter (e.g. lipid productivity or biomass productivity) by more than 10% versus culturing the cell in a medium free of a nitrogen source external to the cells or organisms. In any embodiment biomass productivity can be evaluated by measuring an increase in the total organic carbon of the cells. Nutrient replete conditions are those where the growth of the cultivated organism is not limited by a lack of any nutrient.

In various embodiments the one or more genetic modification(s) can be made in (i.e. derived from) a cell or organism that is a wild type, parent, or laboratory strain. Laboratory strains are cells or organisms that have been cultured in a laboratory setting for a period of time sufficient for the strain to undergo some adaptation(s) advantageous to growth in the laboratory environment and render the strain distinctive versus a more recently cultured wild-type strain. Laboratory strains nevertheless can be genetically modified as described herein and yield significant desirable characteristics from the genetic modification(s), as described herein. For example, laboratory strains can have higher biomass productivity and/or higher lipid productivity than a wild-type strain. In some embodiments one or more genetic modifications disclosed herein can be performed on a laboratory strain to result in a recombinant algal organism of the invention. In such embodiments the laboratory strain can therefore be a corresponding control algal cell or organism described herein that does not have the genetic modification being considered.

### Methods of Producing Lipid

The invention also provides methods for producing a lipid product. The methods involve performing a genetic modification to an algal organism described herein, and culturing the recombinant algal cell or organism described herein to thereby produce a composition containing a lipid product. Any of the methods can also involve a step of harvesting lipid produced by the recombinant algal cell or organism. The culturing can be for a suitable period of time, for example, at least 1 day or at least 3 days or at least 5 days.

The invention also provides methods for producing a composition containing lipids. The methods involve culturing a recombinant algal cell or organism described herein to thereby produce a composition containing lipids. The composition can be a biomass composition. The cultivating can be done in any suitable medium conducive to algal growth (e.g. an algal growth medium or any medium described herein). The methods can also involve a step of harvesting lipids from the composition or biomass containing lipids. The methods can involve a step of harvesting lipids from the recombinant cells or organisms. Any of the methods herein can also involve a step of purifying the lipid containing composition to produce a biofuel or biofuel precursor. A biofuel precursor is a composition containing lipid molecules that can be purified into a biofuel.

The invention also provides methods of producing a recombinant algal cell or organism having higher lipid productivity than a corresponding control cell or organism. The methods involve exposing algal cells or organisms to ultraviolet light to produce a recombinant cell or organism described herein that has higher lipid productivity than a corresponding control cell or organism. In one embodiment algal organisms having higher lipid productivity can be identified by contacting the recombinant cells with a stain that identifies lipids (e.g. by BODIPY dye). Optionally methods can include a step of isolating lipids from the recombinant algal organisms. The recombinant alga can be cultivated in any suitable growth media for algae, such as any of those described herein. The uv treatment can involve, for example, subjecting the culture to uv light, or gamma radiation, or both, for a suitable period of time or under a suitable uv regimen or gamma radiation regimen. Persons of ordinary skill understand suitable regimens for uv exposure for mutagenesis. The uv regimen can involve exposing the cells or organisms to uv radiation, which can be performed in batches with each batch receiving a dose. Multiple cell batches can receive different doses of energy for each batch of cells. For example 4 or 5 batches of cells can receive doses of exposure to 16-57 uJ/cm2 of energy, and exposure energy can increase with each separate batch. The cell batches can be pooled together after exposures are complete. The recombinant alga (or pooled algae) can be cultivated for at least 2 days or at least 3 days, or at least 4 days, or at least 5 days, or at least 6 days, or at least 10 days, or at least 20 days, or from 2-10 days, or from 2-20 days or from 2-25 days after exposure. The recombinant algal organisms can be any described herein.

Any of the recombinant cells or organisms of the invention can be cultivated in batch, semi-continuous, or continuous culture to produce the higher biomass productivity and/or higher lipid productivity. In some embodiments the culture medium can be nutrient replete, or nitrogen deplete (—N). In some embodiment the culturing is under photoautotrophic conditions, and under these conditions inorganic carbon (e.g., carbon dioxide or carbonate) can be the sole or substantially the sole carbon source in the culture medium.

The invention also provides a biofuel comprising a lipid product of any of the recombinant cells or organisms described herein. The biofuel is produced by purifying a lipid containing composition produced by a recombinant algal cell or organism described herein.

The methods disclosed herein can product a biomass product containing a recombinant algal described herein.

### FAME and TOC Analysis Methods

The lipid productivity of the cells or organisms can be measured by any method accepted in the art, for example as an increase or decrease in fatty acid methyl esters comprised in the cell, i.e. FAME analysis. In some embodiments any of the recombinant algal cells or organisms of the invention can have higher biomass productivity as described herein versus corresponding control cells or organisms. In some embodiments the recombinant algal cells or organisms of the invention can have higher lipid productivity and also higher biomass productivity compared to a corresponding control cell or organism. Biomass productivity can be measured by any methods accepted in the art, for example by measuring the total organic carbon (TOC) content of a cell. Embodiments of both methods are provided in the Examples.

“FAME lipids” or “FAME” refers to lipids having acyl moieties that can be derivatized to fatty acid methyl esters, such as, for example, monoacylglycerides, diacylglycerides, triacylglycerides, wax esters, and membrane lipids such as phospholipids, galactolipids, etc. In some embodiments lipid productivity is assessed as FAME productivity in milligrams per liter (mg/L), and for algae, may be reported as grams per square meter per day (g/m2/day). In semi-continuous assays, mg/L values are converted to g/m2/day by taking into account the area of incident irradiance (the SCPA flask rack aperture of 1½ inches×3⅜″, or 0.003145 m2) and the volume of the culture (550 ml). To obtain productivity values in g/m2/day, mg/L values are multiplied by the daily dilution rate (30%) and a conversion factor of 0.175. Where lipid or subcategories thereof (for example, TAG or FAME) are referred to as a percentage, the percentage is a weight percent unless indicated otherwise. The term “fatty acid product” includes free fatty acids, mono-di, or tri-glycerides, fatty aldehydes, fatty alcohols, fatty acid esters (including, but not limited to, wax esters); and hydrocarbons, including, but not limited to, alkanes and alkenes).

In some embodiments the recombinant algal organisms of the invention can have a higher FAME/TOC ratio than a corresponding control organism. In various embodiments the FAME/TOC ratio of the recombinant algal organisms of the invention can be at least 0.4 after two days of cultivation, which cultivation can be batch, continuous, or semi-continuous.

### EMBODIMENTS

In one embodiment the invention provides a recombinant algal organism of the Class Trebouxiophyceae having a genetic modification in a gene or nucleic acid sequence encoding a kinase-like domain described herein. The recombinant alga exhibits higher lipid productivity and/or biomass productivity versus a corresponding control algal cell not having the genetic modification. In various embodiment the Trebouxiophyceae organism can be from the family Oocystaceae or Chlorellaceae. In one embodiment the organism is of the genus Oocystis.

In one embodiment the invention provides a recombinant Trebouxiophyceae organism having a deletion, disruption, or inactivation in a gene or nucleic acid sequence encoding a kinase-like domain described herein. In one embodiment the deletion, disruption, or inactivation involves the insertion of a nonsense mutation in a gene or nucleic acid sequence encoding a kinase-like domain. In one embodiment the kinase-like domain can have at least 80% or at least 90% or at least 95% sequence identity to SEQ ID NO: 1 or 9; or the protein kinase-like domain can be encoded by a nucleic acid sequence having at least 80% or at least 85% or at least 90% or at least 95% sequence identity to SEQ ID NO: 2-3 or 8. The recombinant alga exhibits higher lipid productivity and/or biomass productivity versus a corresponding control algal cell not having the genetic modification. The alga can be a Trebouxiophyceae organism from the family Oocystaceae, for example of the genus Oocystis. The increase in lipid productivity can be an increase of at least 30% w/w, or 30-50% or 30-55%. The recombinant cells or organisms can, optionally, also have an increase in biomass productivity of at least 18% or at least 20% or at least 25%, or 18-40%. Thus in one embodiment the recombinant cells or organisms have an increase in lipid productivity of 30-55% and an increase in biomass productivity of at least 20%. In another embodiment the increase in lipid productivity can be at least 18%. In another embodiment the increase in lipid productivity can be 30-35% and the increase in biomass productivity can be 50-60% or 52-56%. In another embodiment the increase in lipid productivity can be 25-35% or 26-30%, and the increase in biomass productivity can be 40-50% or 42-46%. In another embodiment the increase in lipid productivity can be at least 30%, and the increase in biomass productivity can be at least 50%. In another embodiment the increase in lipid productivity can be at least 25%, and the increase in biomass productivity can be at least 40%.

In one embodiment the invention provides a recombinant Trebouxiophyceae organism having a deletion, disruption, or inactivation in a gene or nucleic acid sequence encoding a kinase-like domain described herein. In one embodiment the deletion, disruption, or inactivation involves the insertion of a nonsense mutation in a gene or nucleic acid sequence encoding the kinase-like domain. In one embodiment the kinase-like domain can have at least 80% or at least 90% or at least 95% sequence identity to SEQ ID NO: 1 or 9. The recombinant alga exhibits higher lipid productivity and/or biomass productivity versus a corresponding control algal cell not having the genetic modification. The alga can be a Trebouxiophyceae organism from the family Oocystaceae, for example of the genus Oocystis. The increase in lipid productivity can be an increase of at least 30% w/w, or 30-33% or 30-35%. The recombinant cells or organisms can, optionally, also have an increase in biomass productivity of at least 25% or at least 30% or 25-35%. In another embodiment the increase in lipid productivity can be at least 18%.

In one embodiment the invention provides a recombinant algal organism of the family Oocystaceae having a deletion, disruption, or inactivation in a gene or nucleic acid sequence encoding a kinase-like domain, which optionally can be SEQ ID NO: 2 or 3, or a variant of either having at least 80% or at least 85% or at least 90% or at least 95% sequence identity to SEQ ID NO: 2-3 or 8. The deletion can be a functional deletion. In one embodiment the deletion, disruption, or inactivation can be a nonsense mutation in SEQ ID NO: 2-3 or 8, or a variant of either. In one embodiment the organism can be of the genus Oocystis. The recombinant alga exhibits higher lipid productivity and/or biomass productivity versus a corresponding control algal cell not having the genetic modification.

In one embodiment the invention provides a recombinant algal organism of the Class Trebouxiophyceae having a genetic modification to a gene or nucleic acid sequence encoding a kinase-like domain. In one embodiment the gene or nucleic acid sequence is that of that SEQ ID NO: 2-3 or 8, or a variant of any of them. The genetic modification can be a deletion (optionally a functional deletion) or disruption of the gene or nucleic acid sequence. The recombinant alga exhibits higher lipid productivity and, optionally, higher biomass productivity versus a corresponding control algal cell not having the genetic modification. In various embodiment the Trebouxiophyceae organism can be from the family Oocystaceae or Chlorellaceae. In one embodiment the organism is of the genus Oocystis.

### Example 1

This example illustrates the mutagenesis and screening of wild-type cells. Mutagenized Oocystis sp. cells were acclimated to diel growth in culture flasks at a light intensity of about 100 uE and 1% CO2 in urea supplemented minimal medium for a week. The culture was scaled up for 3 days in 500 mL square-bottom flasks, bubbled with 1% CO2 at a maximum irradiance of about 1400 uE under diel conditions, to an OD730 of about 1.0. The culture was then centrifuged at 5000 g for 10 mins and the cell pellets resuspended in nitrogen-free minimal medium to an OD730 of about 0.9. This nitrogen-free culture was then incubated for 48 hrs in square-bottom flasks bubbled with 1% CO2 at a maximum irradiance of ˜1400 uE under diel conditions.

Strain 15 (wild-type Oocystis sp.) cells were acclimated to diel growth on urea supplemented medium. The cells were then mutagenized using uv light at a concentration of 2e6 cell/ml and at 22.4, 33.6, 44.8 and 56 mJ/cm2 in a UV Crosslinker apparatus. Mutagenized cells were allowed to recover in the dark for 48 hours. Cultures were scaled up in low light (about 100 uE) before enrichment.

Mutagenized cells were acclimated to diel growth in culture flasks at a light intensity of about 100 uE and 1% CO2 in urea supplemented minimal medium for a week. The culture was scaled up for 3 days 500 mL square-bottom flasks, bubbled with 1% CO2 at a maximum irradiance of about 1400 uE under diel conditions, to an OD730 of about 1.0. The culture was then centrifuged at 5000 g for 10 mins and the cell pellets resuspended in nitrogen-free minimal medium to an OD730 of about 0.9. This nitrogen-free culture was then incubated for 48 hrs under the same conditions.

After 48 hours of nitrogen-free batch growth, an aliquot of cells was removed and subjected to staining with the lipid-specific dye BODIPY for 10 minutes in the dark at a final concentration of 0.2 ug/ml. Mutant cells with the highest level of BODIPY staining were enriched by fluorescence activated cell sorting (FACS). Enriched cell populations were starved for nitrogen as above and subjected to further BODIPY-based FACS enrichment. This iterative process was repeated for a total of five rounds retaining the top cells in each round. The final cells were plated on minimal medium agar plates supplemented with urea to isolate single axenic colonies.

Isolated mutants were scaled up in tissue culture flasks in minimal medium supplemented with urea, then transitioned to nitrogen-free minimal medium. The lipid and biomass accumulation of isolated mutants were compared to the parental strain wild-type cells (Strain 15) with lipid content measured by total fatty acid methyl ester (FAME) analysis and biomass measured by total organic carbon (TOC). As shown in FIG. 1a-c, one mutagenized strain from the screen (STR27560) showed an increase in accumulated FAME and TOC at 2 days in nitrogen deplete minimal medium, as well as an increase in TOC accumulation and an increase in FAME/TOC ratio—an indicator of how much fixed carbon is partitioned to lipids. The results indicated that the isolated mutant STR27560 exhibited improved lipid productivity over the parental wild-type strain. Proline F/2 algae food was used as the nitrogen deplete medium and was made by adding 1.3 ml PROLINE® F/2 Algae Feed Part A (Pentair Aquatic Eco-Systems, Inc., Cary, NC) and 1.3 ml ‘Solution C’ to a final volume of 1 liter of a solution of aquarium salts (17.5 g/L). Solution C is 38.75 g/L NaH2PO4 H2O, 758 mg/L Thiamine HCl, 3.88 mg/L vitamin B12, and 3.84 mg/L biotin. However, persons of ordinary skill in the art with reference to the present disclosure will realize that many algae foods or media can be used with the nitrogen content minimized, such as by omitting urea or available nitrates.

### Example 2

A list of the mutations were identified within exons or at splice junctions in the mutated strain (STR27560). The mutations are identified and set out in FIG. 2. To identify which mutation(s) cause the high lipid phenotype independent knockouts of genes bearing SNPs in the mutated strain were conducted via RNP-based Cas9-mediated gene disruption using biolistic transformation in the wild-type (STR00015) parental strain. All the strains generated were tested for improved biomass and lipid accumulation during nitrogen starvation in T25 flasks.

In one gene encoding a protein kinase-like (PKL) domain, a mutation causing a frameshift in a glycine residue at position 325 (Gly325) led to an altered downstream translation that terminates 100 amino acids downstream of Gly325. The causal link between disrupting PKL function and the improved lipid production observed in STR27560 was tested using RNP-based Cas9-mediated gene disruption targeting the PKL coding sequence. In one Cas9-KO line (named STR30069), a guide RNA (SED ID NO: 4) was designed to target the complementary DNA strand of a sequence region in exon 1 of the PKL coding sequence and transformed into the wild-type parent strain using a gene gun and standard procedures.

DNA sequencing of a first Cas9-KO strain (STR30069) at the locus of the Cas9 targeting site revealed an 88 bp insertion at the Cas9 double stranded break site, which gave rise to a premature stop codon and an 81 amino acid mutant protein (SEQ ID NO: 7), which was deemed be a disruption of the PKL-0924 protein kinase-like domain gene, corresponding to SEQ ID NO: 1 in the original wild-type strain.

In a second Cas9 KO line (named STR30070), a guide RNA (SEQ ID NO: 5) was designed to a sequence region in exon 4 of the coding sequence. DNA sequencing at the locus of the Cas9 targeting site revealed a 220 bp insertion at the Cas9 double stranded break site, which gave rise to a premature stop codon and a 344 mutant amino acid mutant protein (SEQ ID NO: 6), deemed also to be a knockout disruption of the PKL-0924 gene, disclosed as variant 3 in FIG. 2.

### Example 3

T25 lipid productivity assay results for STR30069 and STR30070 revealed an increase in FAME and TOC productivity in nitrogen deficient batch culture versus the wild-type strain (STR0015) (FIG. 3a-b). STR30069 and STR30070 showed a 32% and 28% increase in FAME productivity, respectively, and a 54% and 44% increase in TOC productivity, respectively. It was therefore shown that disruption of the gene encoding the PKL-0924 protein kinase-like domain is sufficient to substantially improve lipid and biomass productivity. Amino acid sequence analysis revealed that the PKL-0924 contains a protein kinase-like family domain in the genomic DNA sequence (SEQ ID NO: 3). Domain search tools predicted the PKL activity in the sequence from amino acid residues 176-506 of SEQ ID NO: 1 or nucleotides 526-1518 of SEQ ID NO: 2, which is characterized as a protein kinase catalytic domain; or SEQ ID NO: 8, which is characterized as the wild type genomic DNA of the identified core PKL domain.

BLAST analysis revealed orthologs of PKL-0924 are broadly distributed in green algae and plants. FIG. 4 provides a table of orthologs of PKL-0924 in green algae. The closest matches to the protein are those classed as Yak1 protein kinases which appear to play diverse regulatory roles through protein phosphyorylation in various organisms. In plants, Arabidopsis thaliana genbank accession BAD93822.1 (Arabidopsis genome accession AT5G39580.1) is a close hit.

Oocystis sp., PRT, PKL-0924 kinase-like protein wild-type sequence
SEQ ID NO: 1
Met Arg Asp Pro Ser Gly Ser Ala Thr Arg Pro ThrProAlaArgValHisLeuAlaArg
AspProSerProCysLysProMetProGlnAspPheSerValSerAspGlnGlnGlyAsn
GlyAlaPheAspAlaProAspAlaAlaGlyAlaAlaCysThrThrLeuProArgArgSer
SerGlyAlaHisAspCysArgAspMetValAlaGlnGluHisGlyAspGlyAlaAlaGln
ValAlaGlnProSerGlnThrSerAlaValSerLysMetThrHisGluLeuLeuAlaThr
TyrGlnArgCysGlyGlyGlyValGlyProThrGlnAlaSerAspAlaThrHisAlaLeu
HisGlnLeuGlnGlnAlaAsnGlnAlaProGluGlnArgGlnGlnAlaGlnArgAsnLeu
LeuThrLysProPheLysGlyLeuHisAsnAspGlyHisAspAsnGluAsnTrpAspLeu
IleIleCysValGlyAspGluPheValSerSerSerAsnLeuLysTyrValValIleAsp
LeuLeuGlyGlnGlyThrPheGlyGlnValValArgCysTrpCysAspGlnThrGlnGlu
TyrValAlaValLysValIleLysAsnGlnProAlaTyrTyrGlnGlnAlaArgValGlu
ValGlyLeuLeuGlnTyrLeuAsnArgCysAlaAspAlaAspAspValArgHisIleVal
ArgLeuArgAspTyrPheLeuPheArgAsnHisLeuCysLeuAlaPheGluLeuLeuSer
ValAsnLeuTyrGluLeuIleLysHisAsnGlnPheArgGlyLeuSerAlaGlyLeuVal
ArgValPheIleAlaGlnLeuLeuAspAlaLeuValValLeuArgGluSerArgLeuIle
HisCysAspLeuLysProGluAsnValLeuLeuThrGlyAlaGluSerAlaAspIleLys
ValIleAspPheGlySerAlaCysLeuGluSerLysThrValTyrSerTyrIleGlnSer
ArgPheTyrArgSerProGluValValLeuGlyTyrProTyrAsnValAlaIleAspMet
TrpSerLeuGlyCysMetAlaAlaGluLeuPheLeuGlyLeuProLeuPheProGlyAla
SerGluHisAspLeuLeuSerArgValValGlnAlaValGlyLeuProProLeuTyrLeu
LeuGlnGlyAlaLysHisThrAsnLysTyrPheLysMetValGluArgValValArgLeu
ProSerGlyArgSerGluValValProGluTyrValMetArgThrAlaAlaGluPheGlu
AlaLeuThrGlyLeuLysAlaThrThrGlyLysArgTyrPheSerHisThrArgLeuGln
AspIleIleAsnSerTyrProSerGluGlyAlaGlySerGluLeuArgArgSerLeuLeu
AspPheLeuArgGlyValLeuAspProAspProAlaAlaArgTrpThrProGlnGlnAla
AlaArgHisProPheValThrGlyGlnProPheAlaAlaProPheGlnProAspGlyGlu
ArgThrProProProProGlyTrpAspAlaSerAlaAlaGlyMetLeuSerSerSerAla
GlyHisGlnAlaAlaGlnGlnGlnGlnAlaGlyTrpAsnGlyAlaSerAlaSerProHis
TyrGlySerAlaAlaAlaAlaMetLeuAlaThrSerProHisValGlnAlaHisValAla
AlaMetAlaAlaLeuAlaGlnGlnGlnGlnGlnGlnGlyMetGlyThrProSerGlyLeu
ProSerTyrArgProGlnProValProValProArgAlaGlyProGlyTyrGlyGlyGly
GlyGlyTrpAlaAlaProTyrProHisGlnHisGlnGlnGlnGlnGlnAlaGlySerLeu
ThrGlnGlnTyrLeuGlnGlnGlnGlnGlnGlnAlaGlyGlySerLeuGlyLeuPheSer
ProProGlyLeuGlyProAlaSerLeuMetGlnLeuAspHisLeuSerAlaAlaSerGly
SerPhePheSerProProGlySerLeuValAsnAlaThrGlyArgLeuAlaProAlaPro
GlyAlaAlaArgTyrGlySerTyrGlnProMetProValAspMetGlyPheSerProAla
GlySerLeuSerAlaAsnSerValLeuAlaValAlaIleAlaAlaAlaAsnAlaAlaAla
AlaSerAlaAlaAlaGlnGlnGlnGlnGlnGlnHisGlyValAlaGluGlnLeuArgLeu
GlnTrpGlnGlyAlaAlaValGlyGlyGlyGlyGlyGlyGlyPheProSerGlyLeuAla
ArgMetSerGlyValAlaGlyGlySerTyrSerGlyGlnSerAlaValProMetValGly
SerTyrAlaSerSerLeuProGlyGlySerAlaAlaThrAlaAlaAlaGlyProGlyThr
GlySerAsnProLeuAlaMetLeuAlaAlaGlnGlnAlaAlaGluAspAlaLeuAlaSer
SerLeuArgArgGlySerMetGlyAlaGlyAlaSerGlyGlyTyrGlyAlaGlyPheGly
GlySerAlaAlaAlaThrAlaAlaProAlaAlaAlaAlaAlaAlaAlaGlnGlnProAla
ProValAspMetSerAlaAlaAlaGlySerLeuGlyAlaValLeuGlnGlnLeuGlnAla
ThrGlnAlaAlaAlaGlnAlaGlnGlnPheValAlaAlaAlaAlaGlyGlyGlySerArg
GlyArgThrProProProProAlaAspGlyAlaLeuAlaThrAlaCysAlaAlaAlaAla
AlaGlyAlaValAlaGlyGlyGlyGlySerAlaHisGlnGlnGlnGlnArgHisGluSer
ValSerProSerProGlyAspTrpAspProLeuTyrSerAspAspGlnLeuLeuGluAsp
AspThrProAlaGlnGlnArgThrProProGlnGlnProGlnGlnArgLysGlySerGly
AsnLeuAlaAlaAlaLeuAlaAlaAlaValThrLeuProAlaGlnGlnGlnGlnTrpGln
GlnGlnGlnGlnGlnArgGlnProLeuAlaGlyGlyLeuSerProProAspProAlaLeu
ThrAlaAlaAlaAlaAlaAlaLeuGlyAlaLeuGlnGlnGlnGlnGlnGlnGlnGlnAla
ThrAlaGlyAlaGlyAlaAlaThrLeuAspThrAlaAspLeuValGlyTrpLeuGlnGln
GlnGlnLeuLeuArgAlaLeuArgProProProAlaThrAlaAspAlaArgGlyAlaGly
GlyAlaAlaAlaAlaAlaAlaArgLeuHis***
PKL-0924 kinase-like protein domain wild-type cDNA (exons only), 30070,
Oocystis, DNA, encodes SEQ ID NO: 1
SEQ ID NO: 2
ATGCGCGATCCCAGTGGGAGTGCCACGCGACCTACTCCTGCTCGGGTTCACCTGGCG
GGGACCCCAGCCCATGCAAACCAATGCCCCAGGATTTCAGCGTGTCTGATCAGCAG
GTAACGGCGCCTTTGACGCGCCTGATGCCGCTGGAGCTGCGTGTACGACATTGCCGC
ACGATCTAGTGGCGCTCACGACTGCAGGGACATGGTAGCCCAGGAACACGGAGACG
GGCCGCCCAGGTGGCACAGCCATCCCAGACATCGGCTGTGTCCAGTAGTTCGTGTGT
ATCTTCGCCACGTATCAACGGTGCGGTGGAGGCGTTGGACCGACACAAGCCAGCGA
GCAACGCACGCGTTGCATCAGCTGCAGCAGGCCAATCAAGCACCGGAGCAGCGGCA
CAGGCACAGCGCAATTTGCTCACGAAGCCTTTCAAGGGTCTGCATAACGACGGTCAT
ACAACGAGAACTGGGACTTGATCATATGTGTCGGGGACGAGTTTGTGTCCAGCTCCA
ACCTCAAGTACGTCGTCATCGACTTGCTGGGCCAGGGCACGTTCGGCCAGGTAGTGC
GCTGCTGGTGCGACCAGACGCAGGAGTATGTAGCCGTCAAGGTGATAAAAAATCAG
CCGGCTTACTATCAGCAAGCGCGCGTAGAGGTTGGCCTGTTGCAATACCTCAACCGC
TGTGCGGACGCGGACGACGTCCGGCACATTGTGCGCCTCCGCGACTACTTTTTGTTC
CGTAACCACTTGTGCCTCGCGTTCGAGCTGCTGTCGGTCAACCTGTACGAGCTCATC
AAGCACAACCAGTTCCGTGGGCTGTCTGCGGGGCTCGTGCGCGTGTTCATCGCTCAG
CTGCTTGATGCGCTGGTGGTGCTGCGTGAGTCTCGCCTCATCCACTGCGACCTCAAG
CCGGAGAACGTGCTGCTTACGGGCGCTGAGTCAGCTGACATAAAGGTCATCGACTTT
GGGTCCGCTTGCCTGGAGAGCAAAACGGTGTACAGCTACATCCAGAGCCGCTTCTAT
CGCTCTCCAGAGGTGGTGCTTGGCTACCCGTACAACGTGGCGATCGACATGTGGTCC
CTGGGCTGCATGGCCGCGGAGCTGTTCCTTGGCCTGCCGCTGTTCCCGGGCGCATCG
GAGCACGATTTGTTGTCTCGGGTGGTGCAGGCGGTGGGCCTCCCGCCGCTGTACCTG
CTGCAGGGCGCAAAGCACACCAACAAGTATTTCAAGATGGTGGAGCGCGTGGTGCG
GCTGCCAAGCGGCAGGTCTGAGGTGGTGCCCGAGTATGTGATGCGCACTGCCGCGG
AGTTTGAGGCTCTCACGGGGCTCAAGGCCACCACCGGCAAGCGCTACTTCTCACATA
CGCGCCTGCAGGACATCATCAACTCATACCCTTCAGAGGGCGCGGGCAGCGAGCTG
CGCCGCTCCCTTCTGGACTTCCTTCGGGGCGTGCTGGACCCCGACCCAGCGGCGCGC
TGGACGCCGCAGCAGGCGGCGCGCCACCCGTTTGTGACGGGCCAGCCGTTCGCGGC
GCCGTTCCAGCCAGACGGGGAGCGTACGCCCCCGCCTCCCGGCTGGGACGCCTCCG
CGGCCGGCATGCTGTCCAGCAGTGCTGGGCACCAGGCCGCTCAGCAGCAGCAGGCA
GGGTGGAACGGCGCGTCCGCGTCGCCGCACTATGGCAGCGCGGCGGCGGCGATGCT
CGCCACGTCGCCGCACGTGCAGGCGCATGTGGCGGCCATGGCGGCGCTCGCCCAGC
AGCAGCAGCAGCAGGGCATGGGCACCCCGTCAGGGCTGCCCTCGTACCGCCCGCAG
CCTGTGCCTGTACCGCGTGCGGGGCCCGGCTACGGCGGTGGCGGCGGCTGGGCAGC
GCCGTATCCCCACCAGCACCAGCAGCAGCAGCAGGCCGGCAGCCTTACGCAGCAGT
ACCTGCAGCAGCAGCAGCAGCAGGCAGGCGGCAGCCTGGGCCTGTTCAGCCCGCCC
GGCCTGGGCCCCGCCAGCCTGATGCAGCTCGACCACCTGAGCGCGGCCTCGGGCTC
GTTCTTCAGCCCGCCTGGGTCGCTGGTCAACGCGACCGGGCGGCTGGCGCCAGCGCC
GGGGGCCGCGCGCTACGGCAGCTACCAGCCGATGCCGGTGGACATGGGCTTCAGCC
CCGCCGGCAGCCTGTCGGCAAACTCGGTGCTGGCGGTGGCCATCGCGGCAGCCAAC
GCCGCAGCCGCCTCGGCCGCTGCGCAGCAGCAGCAGCAGCAGCACGGCGTCGCGGA
GCAGCTGCGGTTGCAGTGGCAGGGCGCCGCCGTGGGCGGCGGCGGCGGCGGCGGCT
TCCCCAGTGGGCTGGCGCGCATGTCGGGCGTGGCAGGGGGCTCGTACTCCGGCCAG
TCTGCAGTGCCGATGGTGGGGTCGTATGCCAGCAGCCTGCCGGGGGGCAGCGCCGC
CACCGCGGCGGCGGGCCCAGGCACGGGCTCAAACCCGCTGGCCATGCTCGCGGCGC
AGCAGGCGGCCGAGGACGCACTCGCCAGCAGCCTGCGGCGCGGGTCGATGGGCGCA
GGCGCGAGCGGCGGGTATGGCGCCGGCTTCGGCGGCTCCGCAGCGGCCACGGCCGC
TCCCGCCGCCGCCGCGGCGGCGGCCCAGCAGCCTGCGCCGGTGGACATGTCGGCTG
CTGCAGGCAGCCTCGGCGCGGTGCTGCAGCAGCTGCAGGCGACACAGGCCGCGGCG
CAGGCGCAACAGTTTGTGGCGGCGGCCGCCGGTGGCGGGAGCCGCGGCCGTACGCC
GCCGCCGCCGGCGGACGGTGCGCTTGCCACCGCTTGCGCTGCAGCTGCTGCGGGAG
CAGTGGCCGGGGGGGGCGGCAGCGCCCACCAGCAGCAGCAGCGGCACGAGTCGGT
GTCGCCATCTCCTGGGGACTGGGACCCGCTGTACAGCGACGACCAGCTCCTGGAGG
ACGATACGCCCGCTCAGCAGCGTACACCGCCGCAACAACCGCAGCAGCGCAAGGGC
AGCGGCAACTTGGCAGCGGCGCTGGCAGCGGCCGTCACGCTGCCTGCCCAGCAGCA
GCAGTGGCAGCAGCAGCAGCAGCAGCGGCAGCCCCTCGCGGGGGGCTGTCTCCGC
CAGACCCTGCGCTAACAGCGGCAGCCGCGGCGGCACTTGGCGCGCTGCAGCAGCAG
CAGCAGCAGCAGCAGGCCACGGCAGGTGCCGGTGCGGCCACACTGGACACCGCAG
ACCTAGTGGGCTGGCTGCAGCAGCAGCAGCTGCTGCGCGCGCTGCGCCCGCCTCCG
GCAACGGCAGACGCCCGCGGCGCCGGAGGTGCCGCCGCTGCCGCGGCCGCCTCCAT
TGAGCCTGCTGGTGTGGCGGGTGCCGTGTCGGCGGCGGCGCCGGTGCCCGGCGACC
AACAGCACCGTGCAGCAGGGCCAATGCCGCCCCTCTCTGCTGCCGAGCAGCAGCAA
ACACAGGCCGGTAGCAGCGGGGGCGCTGCTGCCCATGCTGCGGCAGCAGCAGGGGA
AGGTGTTGCCGCCGGCCTGGGCGGCGGTGCTGGTGGCGCCTCTCGGCGGAACAGTTT
TGAGATTGATCGGAGGGGTAGTTTTGAGATTGGCTTTGAGGTTGCTCCAGTCGTGTC
CACAGGCCTGCAGCAGGCGTCGGGAGCGCGCACTGGCGCGGCACCCGCCCACGCCG
GGGGTGGATTCAGCTGCTTGTCTGCGCAGCTCCACGGCAGCAGGCAGCACAAGCAG
CAGCAGCCGGAAGCGGCAGAACATTCCAGCAAGTAG
DNA, PKL-0924 wild type genomic DNA (including introns), Oocystis sp.,
encodes SEQ ID NO: 1
SEQ ID NO: 3
ATGCGCGATCCCAGTGGGAGTGCCACGCGACCTACTCCTGCTCGGGTTCACCTGGCG
CGGGACCCCAGCCCATGCAAACCAATGCCCCAGGATTTCAGCGTGTCTGATCAGCA
GGGTAACGGCGCCTTTGACGCGCCTGATGCCGCTGGAGCTGCGTGTACGACATTGCC
GCGACGATCTAGTGGCGCTCACGACTGCAGGGACATGGTAGCCCAGGAACACGGAG
ACGGGGCCGCCCAGGTGGCACAGCCATCCCAGACATCGGTGAGAAGACGCCCGGCA
TGCCTATCTTATCGCTTGCGTGTGCGCCAGCGCGTCGCAGTGCCCTGGTTGGGTGCTC
GTCGCGCTGTGACTATCGGTACTTGAGCGTTTTATAACCGGCACGCACGTGCTTGTC
GTTGATGCGTTGTGTAGGCTGTGTCCAGTAGTTCGTGTGTCATCTTCGCCACGTATCA
ACGGTGCGGTGGAGGCGTTGGACCGACACAAGCCAGCGACGCAACGCACGCGTTGC
ATCAGCTGCAGCAGGCCAATCAAGCACCGGAGCAGCGGCAGCAGGCACAGCGCAA
TTTGCTCACGAAGCCTTTCAAGGGTCTGCATAACGACGGTCATGACAACGAGAACTG
GGACTTGATCATATGTGTCGGGGACGAGTTTGTGTCCAGCTCCAACCTCAAGTAAGT
CCACCCCCATCAACCTCTTAACCAACTCTGCCTAGCGTTGTCAAAGTGCTTATACAT
GCAGCGCGCCGCAACGCGACGAGCTTATTCGCTCGTTAAGTAAATTTTTCTGCTGCT
CGCATCGTATCGGTTAACCATCCGATAGCTGACTGCTTCCCTTTCTCTGTATCGACGG
GCGCAGGTACGTCGTCATCGACTTGCTGGGCCAGGGCACGTTCGGCCAGGTAGTGC
GCTGCTGGTGCGACCAGACGCAGGAGTATGTAGCCGTCAAGGTGATAAAAAATCAG
CCGGCTTACTATCAGCAAGCGCGCGTAGAGGTTGGCCTGTTGCAATACCTCAACCGC
TGTGCGGACGCGGACGACGTCCGGCACATTGTGCGCCTCCGCGACTACTTTTTGTTC
CGTAACCACTTGTGCCTCGCGTTCGAGCTGCTGTCGGTCAACCTGTACGAGCTCATC
AAGCACAACCAGTTCCGTGGGCTGTCTGCGGGGCTCGTGCGCGTGTTCATCGCTCAG
GTGAGCACAGGAATGGATTAGCAGCACACCGCCAAGTTGTGCAGTGGCGTTTGGCG
GGGACGCTCACGAGCAGTATCAGCAGATAGGCACGAAGCTAGCCAACTTCAAGCGG
GTTGGAACCTGCCCGCCTGCAGCAGTATGCCTGCCTTGCCCTCCCGCTGTCGTATCG
GCGCGTTCACCCCTGCATCCTGCACCTCCCACCTATGAGTCTGCACCTCCCAAACCC
CTCACACTCCTCTCTCTCTCTCTCTCTCTCTCTCTCTCTCTCTCTCTCTCTCTCTCTCTC
TCTCGCGTGCTATCCCCTCCCTTCACATACGCACACATGCACGCGCATACACGATGC
CACACACACACACACACACACACACACACACACACACACACACACACACACACACA
CACGCACACATGCACTGCAACCTGTCACATGCACGTGCGTGCATGTGCAGCTGCTTG
ATGCGCTGGTGGTGCTGCGTGAGTCTCGCCTCATCCACTGCGACCTCAAGCCGGAGA
ACGTGCTGCTTACGGGCGCTGAGTCAGCTGACATAAAGGTCATCGACTTTGGGTCCG
CTTGCCTGGAGAGCAAAACGGTGTACAGCTACATCCAGAGCCGCTTCTATCGCTCTC
CAGAGGTGCGCGCGCGCGGCCATGGGTCGACATATGGAGTTGGGCTTTGGAAACTG
CTGGCACTGACAGGCTTCGGGACCACCACGGGGCTGTTGTTGTTCGGCAGCGTGGTG
CCTTCAGGGGCGGTGGGGTGGGAGCCTGGTGCTGGGAGCAGGGGAGGCAGAGCTGA
TTCCAGTGGGCAGACCCGATTCCAGTGCTCGCCGCAAGGCGACGGCTACAGCTGGT
GGCTGTTGCGCGAGTTGCAAATGCATGTGCATGTGCACGTGAAGCGCACGCCCACG
CGTAAGCATGGCACACGCGCGCACGTAGACACGCACGCACGCACATACCATATCAC
CACCAACTCCCAAAGGTGGTGCTTGGCTACCCGTACAACGTGGCGATCGACATGTG
GTCCCTGGGCTGCATGGCCGCGGAGCTGTTCCTTGGCCTGCCGCTGTTCCCGGGCGC
ATCGGAGCACGATTTGTTGTCTCGGGTGGTGCAGGCGGTGGGCCTCCCGCCGCTGTA
CCTGCTGCAGGGCGCAAAGCACACCAACAAGTATTTCAAGGTGCGAAAGAGTGCGT
GGGGCGGGCACGCCGTCGATGCGTTGCCACGCCGAGGCTGGCACTTTCTGAATGCC
GAAGCAGTCAGCCATAAGCTTACCTGAAAGGGATGAATGGGGAAGCTGGTGGCCCG
AGCGCACTGACAACCACCTGTGCGGTGAAGGTTGCGTGTGCGCATGCGCGTGTGGG
TGCGTGGAGGGCACAGGTGCGTGCAAGACAGGGGTGGGGTGGGGGGCAGTGCAGT
GCAAGGCCTTGCCTGCGCCCTCGCCACGTCACGTCACCCCACCCAACCCAACTTCGT
CACCGCTTCGTATGCACACGCATGCACAGACACACGCACTGAATTCAAACGCATGT
GCGCATGTTTCAGATGGTGGAGCGCGTGGTGCGGCTGCCAAGCGGCAGGTCTGAGG
TGGTGCCCGAGTATGTGATGCGCACTGCCGCGGAGTTTGAGGCTCTCACGGGGCTCA
AGGCCACCACCGGCAAGCGCTACTTCTCACATACGCGCCTGCAGGTGGGCGGCGTC
AAGTGTCTCATGCGTGTGGCACTGTGTGTGTATGACGCTGTGTATGCGTGCATGCGT
GTGTGCGTTCATGTGTGTGTGTGTGCGCGCGCGTGTGCGTGCGTGCGTGCGTGCGTG
GGTACATGCACGGGTACACGTGTGCATTTTTGTGCGTGTTTGTGTGTGTGTGTGACGT
GCTGTTCCCTGCTTGTAATGCAGCTGAAAGGGGCGCTCATGAGGGGTAGACGATCG
GCGCACTGTGACGCCGCGCTCCTTGTCTTAGAAATCGCGCAGCGGTGGACCTCCTTT
GTGCAGCGCTTGAAACAACGTGCCACTCGCCACCTCGCTGAGACTGGATTTCCATTG
TCACAACTCCCTCAGTGACCCGTGACCCGCCAGACACGACACCCGACCTGGCGGCA
CCCCGCGCGACTCACCCACGCGCACCACCAACGTCGCCTTTGCCAACGTTGCGTTCG
TATCCACACCCTACCTGATGGCCATCACCGCGCGCATGCATGGCGAAGGGAGCTAA
TCCAAGCCACCTCACCCTGCTCCCCACAGGACATCATCAACTCATACCCTTCAGAGG
GCGCGGGCAGCGAGCTGCGCCGCTCCCTTCTGGACTTCCTTCGGGGCGTGCTGGACC
CCGACCCAGCGGCGCGCTGGACGCCGCAGCAGGCGGCGCGCCACCCGTTTGTGACG
GGCCAGCCGTTCGCGGCGCCGTTCCAGCCAGACGGGGTGAGAGGGGAGGGGCTGCT
GGGCGGGGTGAGATGGGAGGGGGGGGGGGGCTGCTGGGCGGGGTGAGATGGGAGG
GGGGGGCTGCTGGGCGGGGTGAGATGGGAGGGGGGGGCTGCTGGGCGGGGTGAGA
GGGGAGGGAGGGGCTGCTGGGCGGGGTGAGAGGGGAGGGAGGGGCTGCTGGGCGG
GGTGAGAGGGGAGGGAGGGGCTGCTGGGCGGGGTGAGAGGGGGGGAGGGGGGGG
CGGCTGGGCGGGGTGAGAGGGGCCTGCTGGGCGGGGTGAGGGGAGGGGGGGGGCC
TGCTGGGCGGGGTGAGAGGGGAGGGGGGGGGGGCTGCTGTGGAACTGCCAGGCCG
TGGGTAGCAGCGTGTGTGTTGTCTGCGGTGCTTGCGGCTGTGTAAGGGCAAGCGAGA
GGGTGCACTTATGTCTTCGGTGCTCCTCGCACGGGACGCACACGCGCGCAAGCATGG
TGCAAACTTACCTACCAGCTGGCCAAGCGTAACGTCATGCTGCCCTAAGCATGGCGG
CCCCTATCTGCTACTGCTGCTATATCCCGTCCCTGCAGGAGCGTACGCCCCCGCCTCC
CGGCTGGGACGCCTCCGCGGCCGGCATGCTGTCCAGCAGTGCTGGGCACCAGGCCG
CTCAGCAGCAGCAGGCAGGGTGGAACGGCGCGTCCGCGTCGCCGCACTATGGCAGC
GCGGCGGCGGCGATGCTCGCCACGTCGCCGCACGTGCAGGCGCATGTGGCGGCCAT
GGCGGCGCTCGCCCAGCAGCAGCAGCAGCAGGGCATGGGCACCCCGTCAGGGCTGC
CCTCGTACCGCCCGCAGCCTGTGCCTGTACCGCGTGCGGGGCCCGGCTACGGCGGTG
GCGGCGGCTGGGCAGCGCCGTATCCCCACCAGCACCAGCAGCAGCAGCAGGCCGGC
AGCCTTACGCAGCAGTACCTGCAGCAGCAGCAGCAGCAGGCAGGCGGCAGCCTGGG
CCTGTTCAGCCCGCCCGGCCTGGGCCCCGCCAGCCTGATGCAGCTCGACCACCTGAG
CGCGGCCTCGGGCTCGTTCTTCAGCCCGCCTGGGTCGCTGGTCAACGCGACCGGGCG
GCTGGCGCCAGCGCCGGGGGCCGCGCGCTACGGCAGCTACCAGCCGATGCCGGTGG
ACATGGGCTTCAGCCCCGCCGGCAGCCTGTCGGCAAACTCGGTGCTGGCGGTGGCC
ATCGCGGCAGCCAACGCCGCAGCCGCCTCGGCCGCTGCGCAGCAGCAGCAGCAGCA
GCACGGCGTCGCGGAGCAGCTGCGGTTGCAGTGGCAGGGCGCCGCCGTGGGCGGCG
GCGGCGGCGGCGGCTTCCCCAGTGGGCTGGCGCGCATGTCGGGCGTGGCAGGGGGC
TCGTACTCCGGCCAGTCTGCAGTGCCGATGGTGGGGTCGTATGCCAGCAGCCTGCCG
GGGGGCAGCGCCGCCACCGCGGCGGCGGGCCCAGGCACGGGCTCAAACCCGCTGGC
CATGCTCGCGGCGCAGCAGGCGGCCGAGGACGCACTCGCCAGCAGCCTGCGGCGCG
GGTCGATGGGCGCAGGCGCGAGCGGCGGGTATGGCGCCGGCTTCGGCGGCTCCGCA
GCGGCCACGGCCGCTCCCGCCGCCGCCGCGGCGGCGGCCCAGCAGCCTGCGCCGGT
GGACATGTCGGCTGCTGCAGGCAGCCTCGGCGCGGTGCTGCAGCAGCTGCAGGCGA
CACAGGCCGCGGCGCAGGCGCAACAGTTTGTGGCGGCGGCCGCCGGTGGCGGGAGC
CGCGGCCGTACGCCGCCGCCGCCGGCGGACGGTGCGCTTGCCACCGCTTGCGCTGC
AGCTGCTGCGGGAGCAGTGGCCGGGGGGGGCGGCAGCGCCCACCAGCAGCAGCAG
CGGCACGAGTCGGTGTCGCCATCTCCTGGGGACTGGGACCCGCTGTACAGGTACTGC
CTCCCACCACCTTCACCCGTTCACGCCACTGCCATGCCTCCATCTGCGTGCCTCAGGC
TGCCGTCGGGACGCGCACATACATGTGTGCTGCAAGGGGCTGGCTAGTCCAAGCGT
AAACGTCATTACTGATGCTTGTACCACTCAATGGATCGTATGACTACTACGTCAGAC
AATACACCCCCAGGCGCTGTTCCCATACCCGTAAGCCTGACATTCAAACGGGATGCT
GACGTTGCCGCACTCCCTCTTCCTCTTTCCCCACCCCACAGCGACGACCAGCTCCTG
GAGGACGATACGCCCGCTCAGCAGCGTACACCGCCGCAACAACCGCAGCAGCGCAA
GGGCAGCGGCAACTTGGCAGCGGCGCTGGCAGCGGCCGTCACGCTGCCTGCCCAGC
AGCAGCAGTGGCAGCAGCAGCAGCAGCAGCGGCAGCCCCTCGCGGGCGGGCTGTCT
CCGCCAGACCCTGCGCTAACAGCGGCAGCCGCGGCGGCACTTGGCGCGCTGCAGCA
GCAGCAGCAGCAGCAGCAGGCCACGGCAGGTGCCGGTGCGGCCACACTGGACACC
GCAGACCTAGTGGGCTGGCTGCAGCAGCAGCAGCTGCTGCGCGCGCTGCGCCCGCC
TCCGGCAACGGCAGACGCCCGCGGCGCCGGAGGTGCCGCCGCTGCCGCGGCCGCCT
CCATTGAGCCTGCTGGTGTGGCGGGTGCCGTGTCGGCGGCGGCGCCGGTGCCCGGC
GACCAACAGCACCGTGCAGCAGGGCCAATGCCGCCCCTCTCTGCTGCCGAGCAGCA
GCAAACACAGGCCGGTAGCAGCGGGGGCGCTGCTGCCCATGCTGCGGCAGCAGCAG
GGGAAGGTGTTGCCGCCGGCCTGGGCGGCGGTGCTGGTGGCGCCTCTCGGCGGAAC
AGTTTTGAGATTGATCGGAGGGGTAGTTTTGAGATTGGCTTTGAGGTTGCTCCAGTC
GTGTCCACAGGTGGGCCCTGTCCCTCACCTAACTGTTCATCTCCCTCCAGCCATTGCA
CGTGCTGCCGTATGTTGCATACCCGCTTCCAAGTATCGAGTGCTGCGTGCCCGTTCA
AGTGGGTTTCTAGTGCTTCCGCGTTCTGGTTGGTAGCACAGCAGCCTGCTTGCAAGG
TGCCACGTCGGCTCTCAACACACTAATGATTCGCTGCATTACTCTTGCCTCTGCCGGT
GTGACTTCGCAGGCCTGCAGCAGGCGTCGGGAGCGCGCACTGGCGCGGCACCCGCC
CACGCCGGGGGTGGATTCAGCTGCTTGTCTGCGCAGCTCCACGGCAGCAGGCAGCA
CAAGCAGCAGCAGCCGGAAGCGGCAGAACATTCCAGCAAGTAG
artificial sequence, DNA, encodes gRNA for target in exon 1 SEQ ID NO: 1
SEQ ID NO: 4
TGAGCGCCACTAGATCGTCGCGG
artificial sequence, DNA, encodes gRNA for target in exon 4 SEQ ID NO: 1
SEQ ID NO: 5
PKL-0924 kinase-like protein mutated sequence, Oocystis sp., PRT
SEQ ID NO: 6
MetArgAspProSerGlySerAlaThrArgProThrProAlaArgValHisLeuAlaArg
AspProSerProCysLysProMetProGlnAspPheSerValSerAspGlnGlnGlyAsn
GlyAlaPheAspAlaProAspAlaAlaGlyAlaAlaCysThrThrLeuProArgArgSer
SerGlyAlaHisAspCysArgAspMetValAlaGlnGluHisGlyAspGlyAlaAlaGln
ValAlaGlnProSerGlnThrSerAlaValSerLysMetThrHisGluLeuLeuAlaThr
TyrGlnArgCysGlyGlyGlyValGlyProThrGlnAlaSerAspAlaThrHisAlaLeu
HisGlnLeuGlnGlnAlaAsnGlnAlaProGluGlnArgGlnGlnAlaGlnArgAsnLeu
LeuThrLysProPheLysGlyLeuHisAsnAspGlyHisAspAsnGluAsnTrpAspLeu
IleIleCysValGlyAspGluPheValSerSerSerAsnLeuLysTyrValValIleAsp
LeuLeuGlyGlnGlyThrPheGlyGlnValValArgCysTrpCysAspGlnThrGlnGlu
TyrValAlaValLysValIleLysAsnGlnProAlaTyrTyrGlnGlnAlaArgValGlu
ValGlyLeuLeuGlnTyrLeuAsnArgCysAlaAspAlaAspAspValArgHisIleVal
ArgLeuArgAspTyrPheLeuPheArgAsnHisLeuCysLeuAlaPheGluLeuLeuSer
ValAsnLeuTyrGluLeuIleLysHisAsnGlnPheArgGlyLeuSerAlaGlyLeuVal
ArgValPheIleAlaGlnLeuLeuAspAlaLeuValValLeuArgGluSerArgLeuIle
HisCysAspLeuLysProGluAsnValLeuLeuThrGlyAlaGluSerAlaAspIleLys
ValIleAspPheTrpPheArgLeuProGlyGluGlnAsnGlyValGlnLeuHisProGlu
ProLeuLeuSerLeuSerArgGlyGlyAlaTrpLeuProValGlnArgGlyAspArgHis
ValValProGlyLeuHisGlyArgGlyAlaValProTrpProAlaAlaValProGlyArg
IleGlyAlaArgPheValValSerGlyGlyAlaGlyGlyGlyProProAlaAlaValPro
AlaAlaGlyArgLysAlaHisGlnGlnValPheGlnAspGlyGlyAlaArgGlyAlaAla
AlaLysArgGlnVal**
PRT, PKL-0924 kinase-like protein mutated sequence variant 30069,
Oocystis sp.
SEQ ID NO: 7
MetArgAspProSerGlySerAlaThrArgProThrProAlaArgValHisLeuAlaArg
AspProSerProCysLysProMetProGlnAspPheSerValSerAspGlnGlnGlyAsn
GlyAlaPheAspAlaProAspAlaAlaGlyAlaAlaCysThrThrLeuProArgGlnGly
ThrAlaThrProArgArgGluIleAspGlyProAlaThrArgAsnLysAlaAlaSerLeu
Arg*
DNA, PKL-0924 kinase-like protein core domain of wild-type genomic DNA
sequence, Oocystis sp.
SEQ ID NO: 8
CCGCGACGATCTAGTGGCGCTCACGACTGCAGGGACATGGTAGCCCAGGAACACGG
AGACGGGGCCGCCCAGGTGGCACAGCCATCCCAGACATCGGCTGTGTCCAGTAGTT
CGTGTGTCATCTTCGCCACGTATCAACGGTGCGGTGGAGGCGTTGGACCGACACAAG
CCAGCGACGCAACGCACGCGTTGCATCAGCTGCAGCAGGCCAATCAAGCACCGGAG
CAGCGGCAGCAGGCACAGCGCAATTTGCTCACGAAGCCTTTCAAGGGTCTGCATAA
CGACGGTCATGACAACGAGAACTGGGACTTGATCATATGTGTCGGGGACGAGTTTG
TGTCCAGCTCCAACCTCAAGTACGTCGTCATCGACTTGCTGGGCCAGGGCACGTTCG
GCCAGGTAGTGCGCTGCTGGTGCGACCAGACGCAGGAGTATGTAGCCGTCAAGGTG
ATAAAAAATCAGCCGGCTTACTATCAGCAAGCGCGCGTAGAGGTTGGCCTGTTGCA
ATACCTCAACCGCTGTGCGGACGCGGACGACGTCCGGCACATTGTGCGCCTCCGCG
ACTACTTTTTGTTCCGTAACCACTTGTGCCTCGCGTTCGAGCTGCTGTCGGTCAACCT
GTACGAGCTCATCAAGCACAACCAGTTCCGTGGGCTGTCTGCGGGGCTCGTGCGCGT
GTTCATCGCTCAGCTGCTTGATGCGCTGGTGGTGCTGCGTGAGTCTCGCCTCATCCAC
TGCGACCTCAAGCCGGAGAACGTGCTGCTTACGGGCGCTGAGTCAGCTGACATAAA
GGTCATCGACTTTGGGTCCGCTTGCCTGGAGAGCAAAACGGTGTACAGCTACATCCA
GAGCCGCTTCTATCGCTCTCCAGAGG
PRT, core domain of PKL-0924 protein kinase-like domain, Oocystis sp.
SEQ ID NO: 9
TyrValValIleAspLeuLeuGlyGlnGlyThrPheGlyGlnValValArgCysTrpCys
AspGlnThrGlnGluTyrValAlaValLysValIleLysAsnGlnProAlaTyrTyrGln
GlnAlaArgValGluValGlyLeuLeuGlnTyrLeuAsnArgCysAlaAspAlaAspAsp
ValArgHisIleValArgLeuArgAspTyrPheLeuPheArgAsnHisLeuCysLeuAla
PheGluLeuLeuSerValAsnLeuTyrGluLeuIleLysHisAsnGlnPheArgGlyLeu
SerAlaGlyLeuValArgValPheIleAlaGlnLeuLeuAspAlaLeuValValLeuArg
GluSerArgLeuIleHisCysAspLeuLysProGluAsnValLeuLeuThrGlyAlaGlu
SerAlaAspIleLysValIleAspPheGlySerAlaCysLeuGluSerLysThrValTyr
SerTyrIleGlnSerArgPheTyrArgSerProGluValValLeuGlyTyrProTyrAsn
ValAlaIleAspMetTrpSerLeuGlyCysMetAlaAlaGluLeuPheLeuGlyLeuPro
LeuPheProGlyAlaSerGluHisAspLeuLeuSerArgValValGlnAlaValGlyLeu
ProProLeuTyrLeuLeuGlnGlyAlaLysHisThrAsnLysTyrPheLysMetValGlu
ArgValValArgLeuProSerGlyArgSerGluValValProGluTyrValMetArgThr
AlaAlaGluPheGluAlaLeuThrGlyLeuLysAlaThrThrGlyLysArgTyrPheSer
HisThrArgLeuGlnAspIleIleAsnSerTyrProSerGluGlyAlaGlySerGluLeu
ArgArgSerLeuLeuAspPheLeuArgGlyValLeuAspProAspProAlaAlaArgTrp
ThrProGlnGlnAlaAlaArgHisProPheVal

Although the invention has been described with reference to the presently preferred embodiment, it should be understood that various modifications can be made without departing from the spirit of the inventions. Accordingly, the invention is limited only by the following claims.

What is claimed is:

1. A recombinant algal organism comprising a deletion, disruption, or inactivation of:
a gene encoding a protein kinase-like domain that has a polypeptide sequence having at least 80% sequence identity to SEQ ID NO: 1; or
a gene encoding a protein kinase-like domain having a nucleic acid sequence having at least 80% sequence identity to SEQ ID NO: 2 or 3;
wherein the recombinant algal organism exhibits higher biomass productivity and higher lipid productivity versus a corresponding control algal organism not having the deletion, disruption, or inactivation.

2. The recombinant algal organism of claim 1, having a gene encoding a protein kinase-like domain that has a polypeptide sequence having at least 85% sequence identity to SEQ ID NO: 1.

3. The recombinant algal organism of claim 1, having a gene encoding a protein kinase-like domain having a nucleic acid sequence having at least 85% sequence identity to SEQ ID NO: 3.

4. The recombinant algal organism of claim 1, wherein the organism is a Chlorophyte alga.

5. The recombinant algal organism of claim 4, wherein the organism is of the Class Trebouxiophyceae.

6. The recombinant algal organism of claim 1, wherein the deletion, disruption, or inactivation is to a regulatory sequence of the gene encoding the protein kinase-like domain.

7. The recombinant algal organism of claim 6, wherein the regulatory sequence is a promoter.

8. The recombinant algal organism of claim 1, wherein the deletion, disruption, or inactivation comprises a deletion of one or more amino acids of the encoded protein kinase-like domain.

9. The recombinant algal organism of claim 1, wherein the deletion, disruption, or inactivation comprises an insertion in the gene encoding the kinase-like protein.

10. The recombinant algal organism of claim 9, wherein the insertion comprises insertion of a stop codon in a sequence encoding the kinase-like domain.

11. The recombinant algal organism of claim 1, wherein the recombinant alga has at least 20% higher lipid productivity versus a control algae.

12. The recombinant algal organism of claim 1, wherein the recombinant alga has at least 25% higher lipid productivity versus a control algae.

13. The recombinant algal organism of claim 1, wherein the recombinant alga has at least 35% higher biomass productivity per unit time versus the corresponding control algal cell or organism.

14. The recombinant algal organism of claim 1, wherein the recombinant alga has a FAME/TOC ratio of at least 0.4 after two days of cultivation.

15. The recombinant algal organism of claim 1, wherein the recombinant alga has higher biomass productivity under nitrogen deficient conditions.

16. The recombinant algal organism of claim 1, wherein the recombinant alga has higher total organic carbon production under nitrogen deficient conditions.

17. The recombinant algal organism of claim 1, of a family selected from the group consisting of: Oocystaceae, Chlorellaceae, and Eustigmatophyceae.

18. The recombinant algal organism of claim 1, wherein the recombinant alga is of a genus selected from the group consisting of: Chlorella, Parachlorella, Picochlorum, Tetraselmis, and Oocystis.

19. The recombinant algal organism of claim 18, wherein the recombinant alga is from the genus Oocystis.

20. The recombinant algal organism of claim 1, wherein the protein kinase-like domain comprises a polypeptide sequence having at least 90% sequence identity to SEQ ID NOs: 1 or 9.

21. The recombinant algal organism of claim 1, wherein the gene encoding the protein kinase-like domain has a nucleic acid sequence having at least 90% sequence identity to SEQ ID NO: 2 or SEQ ID NO: 3.

22. A biomass product comprising the recombinant alga of claim 1.

23. A recombinant algal organism comprising a genetic modification to:
a gene encoding a protein kinase-like domain that has a polypeptide sequence having at least 90% sequence identity to SEQ ID NO: 9; or
a gene encoding a protein kinase-like domain having a nucleic acid sequence having at least 90% sequence identity to SEQ ID NO: 8; and
wherein the recombinant algal organism exhibits higher biomass productivity and higher lipid productivity versus a corresponding control algal organism not having the genetic modification.

24. The recombinant algal organism of claim 23, wherein the genetic modification is a deletion, disruption, or inactivation.

25. The recombinant algal organism of claim 23, wherein the recombinant alga has at least 25% higher lipid productivity versus a control algae.

26. The recombinant algal organism of claim 23, wherein the recombinant alga has at least 35% higher biomass productivity per unit time versus the corresponding control algal cell or organism.

27. A method of producing a composition containing lipids comprising performing a genetic modification in an algal organism to:
a gene encoding a protein kinase-like domain having a polypeptide sequence having at least 80% sequence identity to SEQ ID NO: 1 or 9; or
a gene encoding a protein kinase-like domain having a nucleic acid sequence having at least 80% sequence identity to SEQ ID NO: 2-3 or 8;
wherein the recombinant algal organism exhibits higher biomass productivity and higher lipid productivity versus a corresponding control algal organism not having the genetic modification; and
cultivating the organism, and thereby producing a composition containing lipids.

28. The method of claim 27, further comprising harvesting a lipidic composition from the algal organism.

29. The method of claim 27, wherein the genetic modifications to the sequence encoding the protein-kinase like domain is a deletion, disruption, or inactivation.

30. The method of claim 29, wherein the genetic modification is a disruption.

31. The method of claim 27, wherein the recombinant alga has at least 50% greater lipid productivity versus a control alga.
